(19) United States
(12) Patent Application Publication
(10) Pub. No.: US 2026/0174857 A1
(43) Pub. Date: Jun. 25, 2026

(54) METHODS OF ADJUVANT TREATMENT FOR ALLERGY IMMUNOTHERAPY

(71) Applicant: Revelation Biosciences, Inc., San Diego, CA (US)

(72) Inventors: Robin L. Marsden, EI Cajon, CA (US); James M. Rolke, Tucson, AZ (US); Kristine Storey, San Diego, CA (US); Sima Patel, San Diego, CA (US)

(21) Appl. No.: 19/124,696

(22) PCT Filed: Oct. 26, 2023
(86) PCT No.: PCT/US2023/036027

§ 371 Date: Apr. 25, 2025

Related U.S. Application Data
(60) Provisional application No. 63/419,545, filed on Oct. 26, 2022.

(51) Int. Cl. A61K 45/06; A61K 9/00

(52) U.S. Cl. CPC A61K 45/06; A61K 9/0014

(57) ABSTRACT

The present disclosure provides adjuvant treatment in a subject undergoing allergy immunotherapy (AIT) comprising administering to a subject in need thereof a monophosphoryl lipid A (MPLA) compound.

### CROSS-REFERENCE TO RELATED APPLICATIONS

This application claims the benefit of priority to U.S. Provisional Application No. 63/419,545, filed on Oct. 26, 2022, the contents of which are hereby incorporated in their entirety.

### BACKGROUND OF THE INVENTION

Allergic disease (“allergies”) is perhaps the most preeminent area of immunology investigation due to prevalence, relevance as a public health issue, and implications to patient outcomes such as burden of disease. Allergens (e.g., food, pollens, fungi, animals, dust mites, cockroaches, and venom) are non-infectious substances from the environment that affect genetically predisposed individuals. United States population data collected in 2018 estimated that approximately 7.7% and 7.2% of adults and children, respectively, suffered from allergic rhinitis symptoms (hay fever); 9.6% of children suffered from respiratory allergies. Collectively, allergic diseases constitute a spectrum of health severities, making disease management a challenge to surmount.

Allergic reactions begin in susceptible individuals after exposure to a sufficient amount of allergen, also called “antigen”. There are 2 types of clinical distinctions that characterize allergic manifestations in susceptible individuals: IgE mediated (or IgE-dependent) and non-IgE-mediated (or IgE-independent). Allergic rhinitis, asthma, and venom allergies are major IgE-mediated allergy disorders, especially in children (Alvaro-Lozano 2020). Individuals who produce IgE-mediated allergy signs and symptoms have Type I hypersensitivity (1 of 4 types) and are often classified as “atopic” patients due to their genetic predisposition and tendency to develop sensitivities earlier in life.

Allergic mechanisms at the cellular level vary between different antigens. Allergic rhinitis, for example, develops via a process of sensitization, challenge, and elicitation, and encompasses both the innate and adaptive immune systems. Type I hypersensitivity results in activation of a Th2 response toward antigen, resulting in allergen-specific IgE antibody production (Bousquet 2020). Allergen-specific IgE antibodies bind to mast cell and basophil receptors upon subsequent challenge with antigen, resulting in degranulation and release of inflammatory mediators (e.g., histamine, leukotrienes, tryptase). These mediators cause mobilization of basophils, eosinophils, and T lymphocytes to the site of insult, as well as activation of T cells, which compound symptomology and enhance inflammation. Systemic responses, e.g., anaphylaxis, may also occur within minutes or hours post-challenge. Accordingly, there remains a significant need for effective therapies for treating and preventing allergic disease.

TLR4 stimulation leads to activation of the innate immune system via generation of a range of different cytokines, such as pro-inflammatory IL-6, as well as anti-inflammatory IL-10. The character and magnitude of this cytokine response is dependent on the TLR4 agonist, as TLR4 signals though both the MyD88 and TRIF pathway.

Stimulation of TLR4 also results in mobilization of exosomes from epithelia which can release antimicrobial peptides and nitric oxide that can destroy invading pathogens/antigens (Nocera 2018). TLR4 stimulation also leads to generation of Type I interferons (IFNs) which block activation of Th2 cellular activity, resulting in reduced IgE secretion and reduction in allergic symptoms (Gonzalez-van Horn 2015). Type I IFN response results in the generation of IP-10 (CXCL10), which is capable of binding to CCR3, the native receptor for eotaxin and is present on cells involved in the allergic response, including eosinophils, basophils, and mast cells (Loetscher 2001). IP-10 binding to CCR3 prevents eotaxin from binding and recruiting immune cells, thereby reducing recruitment of Th2 cells and attenuating the allergic response.

Use of allergy immunotherapy (AIT) is widespread and well-accepted among immunologists, and its disease modifying mechanism of action supports a favorable benefit-risk profile. The treatment process for immunotherapy involves a build-up phase of frequent dosing of an allergen followed by a maintenance phase once a target treatment is reached. Frequency of AIT depends on the type of allergy and the prescribed schedule. AIT treatments can be as frequent as several times per day (rush schedule), clustered (2 to 3 per visit), or a conventional schedule that follows 1 to 2 injections per week. Peanut AIT has an FDA-approved oral pill option that follows a daily build-up phase. In some patients, it can take as long as 6 months to reach the target dose, after which the maintenance phase may begin, requiring fewer AIT treatments to maintain effect (about every 2 to 4 weeks between treatments). Duration of therapy is individualized; some cases may go into remission while other cases require continued treatment for an indeterminate amount of time (www.aaaai.org/Aaaai/media/MediaLibrary/PDF%20Documents/Practice%20and%20Parameters/Allergen-immunotherapy-Jan-2011.pdf Cox 2011). Compliance to AIT is critical to treatment effectiveness.

Due to the inconvenience, cost, and lengthy time of AIT, there is considerable interest in expediting the build-up treatment course in order to achieve maintenance or remission sooner.

### SUMMARY

The present disclosure provides adjuvant treatment in a subject undergoing allergy immunotherapy (AIT) comprising administering to a subject in need thereof a monophosphoryl lipid A (MPLA) compound. In certain embodiments, the MPLA compound is administered systemically. In more particular embodiments, the MPLA compound is administered intravenously, subcutaneously, intrathecally, orally, sublingually, or intramuscularly. In certain embodiments, the MPLA compound is administered to the mucosa of the subject, for example, intranasally.

In certain embodiments, the MPLA is administered in a dose from about 0.01 mcg to about 10,000 mcg, for example about 1 mcg to about 1000 mcg, about 10 mcg to about 500 mcg, or about 200 mcg.

The MPLA compound may advantageously be administered within 3 days prior to AIT treatment or within 24 hours after AIT treatment course.

In some embodiments, the MPLA compound is selected from phosphorylated hexaacyl disaccharide (PHAD), PHAD-504, 3D (6-acyl)-PHAD, 3D-PHAD and any combination thereof. In preferred embodiments, the MPLA compound is PHAD.

In certain embodiments, the composition further comprises a sugar, such as a monosaccharide, a disaccharide, a trisaccharide, a linear oligosaccharide, a branched oligosaccharide, a cyclic oligosaccharide, a linear polysaccharide, a branched polysaccharide, or any combination thereof. Preferably, the disaccharide is trehalose. Preferably, the cyclic oligosaccharide is beta-cyclodextrin. More preferably, the composition comprises beta-cyclodextrin and trehalose.

In some embodiments, the composition further comprises a surfactant selected from poloxamer 407, poloxamer 181, dodecyltrimethylammonium bromide (DTAB), n-dodecyl octa (ethylene oxide) (C12E8), n-dodecyl tetra (ethylene oxide) (C12E4), dioctanoyl phosphatidylcholine (C8-lecithin), Polyoxyl 35 castor oil, Cremophor EL (CrEL), Octaethylene glycol monododecyl ether (C12E8), hexadecyltrimethylammonium bromide (CTAB), polypropylene oxide (PPO), polyethylene oxide (PEO), PEO-poly(D,L-lactic acid-co-caprolactone) (PEO-PDLLA), sodium dodecyl sulfate (SDS), and any combination thereof.

In some embodiments, the composition further comprises a phospholipid selected from phosphatidic acid (“PA”), phosphatidylcholine (“PC”), phosphatidylglycerol (“PG”), phophatidylethanolamine (“PE”), phophatidylinositol (“PI”), phosphatidylserine (“PS”), sphingomyelin (including brain sphingomyelin), lecithin, lysolecithin, lysophosphatidylethanolamine, cerebrosides, diarachidoylphosphatidylcholine (“DAPC”), didecanoyl-L-alpha-phosphatidylcholine (“DDPC”), dielaidoylphosphatidylcholine (“DEPC”), dilauroylphosphatidylcholine (“DLPC”), dilinoleoylphosphatidylcholine, dimyristoylphosphatidylcholine (“DMPC”), dioleoylphosphatidylcholine (“DOPC”), dipalmitoylphosphatidylcholine (“DPPC”), distearoylphosphatidylcholine (“DSPC”), 1-palmitoyl-2-oleoyl-phosphatidylcholine (“POPC”), diarachidoylphosphatidylglycerol (“DAPG”), didecanoyl-L-alpha-phosphatidylglycerol (“DDPG”), dielaidoylphosphatidylglycerol (“DEPG”), dilauroylphosphatidylglycerol (“DLPG”), dilinoleoylphosphatidylglycerol, dimyristoylphosphatidylglycerol (“DMPG”), dioleoylphosphatidylglycerol (“DOPG”), dipalmitoylphosphatidylglycerol (“DPPG”), distearoylphosphatidylglycerol (“DSPG”), 1-palmitoyl-2-oleoyl-phosphatidylglycerol (“POPG”), diarachidoylphosphatidylethanolamine (“DAPE”), didecanoyl-L-alpha-phosphatidylethanolamine (“DDPE”), dielaidoylphosphatidylethanolamine (“DEPE”), dilauroylphosphatidylethanolamine (“DLPE”), dilinoleoylphosphatidylethanolamine, dimyristoylphosphatidylethanolamine (“DMPE”), dioleoylphosphatidylethanolamine (“DOPE”), dipalmitoylphosphatidylethanolamine (“DPPE”), distearoylphosphatidylethanolamine (“DSPE”), 1-palmitoyl-2-oleoyl-phosphatidylethanolamine (“POPE”), diarachidoylphosphatidylinositol (“DAPI”), didecanoyl-L-alpha-phosphatidylinositol (“DDPI”), dielaidoylphosphatidylinositol (“DEPI”), dilauroylphosphatidylinositol (“DLPI”), dilinoleoylphosphatidylinositol, dimyristoylphosphatidylinositol (“DMPI”), dioleoylphosphatidylinositol (“DOPI”), dipalmitoylphosphatidylinositol (“DPPI”), distearoylphosphatidylinositol (“DSPI”), 1-palmitoyl-2-oleoyl-phosphatidylinositol (“POPI”), diarachidoylphosphatidylserine (“DAPS”), di decanoyl-L-alpha-phosphatidylserine (“DDPS”), dielaidoylphosphatidylserine (“DEPS”), dilauroylphosphatidylserine (“DLPS”), dilinoleoylphosphatidylserine, dimyristoylphosphatidylserine (“DMPS”), dioleoylphosphatidylserine (“DOPS”), dipalmitoylphosphatidylserine (“DPPS”), distearoylphosphatidylserine (“DSPS”), 1-palmitoyl-2-oleoyl-phosphatidylserine (“POPS”), diarachidoyl sphingomyelin, didecanoyl sphingomyelin, dielaidoyl sphingomyelin, dilauroyl sphingomyelin, dilinolcoyl sphingomyelin, dimyristoyl sphingomyelin, sphingomyelin, dioleoyl sphingomyelin, dipalmitoyl sphingomyelin, distearoyl sphingomyelin, 1-palmitoyl-2-oleoyl-sphingomyelin, and any combination thereof.

In some embodiments, the MPLA compound is between 0.5% and 10% by weight of the composition.

In some embodiments, the sugar is between 80% and 99.5% by weight of the composition.

In some embodiments, the surfactant is between 0.5% and 10% by weight of the composition.

In some embodiments, the phospholipid is between 0.5% and 10% by weight of the composition.

In some embodiments, the composition is a dry powder.

In some embodiments, the composition is an aqueous composition.

In certain embodiments, the composition further comprises an organic solvent. The organic solvent and water may be present in a volume to volume ratio of about 1:1500 to about 1:50, about 1:1000 to about 1:100, or preferably about 1:800.

In some embodiments, the organic solvent is miscible with water, such as an alcohol, glycerin, low molecular weight polyethylene glycol, and low molecular weight poloxamers. Preferably, the organic solvent is an alcohol, and more preferably, the organic solvent methanol, ethanol, isopropanol, or t-butanol, and even more preferably, ethanol.

In some embodiments, the composition has an MPLA compound concentration of about 0.01 μg/mL to about 30 mg/ml, about 50 μg/mL to about 500 μg/mL, about 100 μg/mL, or about 200 μg/mL.

In some embodiments, the composition further comprises a stabilizer.

In some embodiments, the composition comprises micelles having an average diameter or length of about 1 nm to about 1000 nm, about 50 nm to about 500 nm, or about 100 nm to about 500 nm.

In some embodiments, the composition further comprises a mucoadhesive agent, such as cellulose derivatives, polyacrylates, a starch, chitosan, glycosylaminoglycans, hyaluronic acid, cellulose derivatives, or any combination thereof.

In some embodiments, the composition further comprises a pH modifier, a pH buffer, an emulsifier, a tonicity modifier, a stabilizer, a preservative, a surfactant, a bulking agent, a flavorant, or any combination thereof.

In some embodiments, the bulking agent is selected from mannitol, trehalose, chitosan, hydroxypropylmethylcellulose (HPMC), dextran, pea starch, and sucrose.

In some embodiments, the composition further comprises liposomes.

In some embodiments, the composition further comprises nanoparticles.

In some embodiments, the composition further comprises microparticles.

The method may advantageously deliver a total dose of from 0.1 to 800 micrograms of MPLA compound to the subject. In other embodiments, the method delivers a dose of from 1 to 800 mcg, 10 to 100 mcg, 25 to 75 mcg or about 50 mcg.

In some embodiments, the MPLA compound is administered within 3 days prior to an AIT treatment. In other embodiments, the MPLA compound is administered within about 24 hours after an AIT treatment course. The AIT treatment may be for a food allergy, animal dander, feathers, pollen, or insect venom. In some embodiments, the dander is selected from horse dander, dog dander, cat dander, and any combination thereof, in some embodiments, the insect venom is hymenoptera venom. In some embodiments, the food allergy is selected from peanut, tree nut, sesame seed, dairy, fish, shellfish, wheat, soy, egg, and any combination thereof. Preferably, the food allergen is peanut.

In other embodiments, the present disclosure provides a method of augmenting the therapeutic action of AIT comprising:
a) determining the subject's response via clinical symptomology using a validated or widely accepted subjective symptom scale
b) determining the subject's immunoglobulin profile in blood, saliva, or nasal specimens
c) determining the subject's immunogenic mediator constituents (e.g., histamine, eotaxins, or leukotrienes) profile in blood, saliva, nasal, or urine specimens
d) determining the subject's immunogenic effector cell (basophils, eosinophils, mast cells) profile in blood, saliva, nasal, or urine specimens
e) determining the subject's cytokine and/or chemokine profiles in blood or nasal specimens; and
f) determining the subject's objective clinical measures such as hemoglobin A1c, peak nasal inspiratory flow, FEV1, etc.

### BRIEF DESCRIPTION OF THE FIGURES

FIG. 1 depicts the structure of synthetic phosphorylated, hexaacyl disaccharide (PHAD).

FIG. 2 IP-10 upregulation as a function of dose using representative formulated PHAD. Multiple preparations of PHAD were assessed in an in vitro cell based activity assay using mouse macrophages of the J774 cell line to demonstrate stimulation of TLR4 in response to PHAD as measured by upregulation of IP-10.

FIG. 3 is a schematic drawing showing the molecular mechanism of the micellular LPS/LBP/CD14 triplex and interaction with TLR4/MD-2 receptor complex.

FIG. 4 Monophosphoryl Lipid A (MPLA) like compounds can preferentially stimulate TLR4 to activate the TRIF pathway leading to the production of protective cytokines.

FIG. 5 PHAD administration preconditions the innate immune system to rapidly respond to a subsequent stress (infection, trauma, etc.) via TLR4 stimulation, facilitating redirection of the innate immune response.

FIG. 6 The stimulation of TLR4 in response to MPLA compounds leads to generation of IL-10 and Type I interferons preferentially through the TRIF pathway (FIGS. 4 and 5). A negative reciprocal feedback loop has been observed to exist between IL-10 and Type I interferon activity on Th2-biased cellular activity.

FIGS. 7a and 7b Administration of two doses of PHAD results in an initial increase in both inflammatory and protective cytokines following a first dose and an attenuated pro-inflammatory response following a second dose at 24 hours (e.g., a smaller increase in pro-inflammatory cytokines (e.g., IL-6) and a larger increase in anti-inflammatory cytokines (e.g., IL-10)).

FIG. 8 Representative structure of lipopolysaccharides obtained from gram-negative bacteria.

FIG. 9 Results of an ad hoc analysis of IP-10 upregulation in response to composition of PHAD in normal healthy human volunteers.

FIG. 10 depicts the structure of monophosphoryl 3-deacyl lipid A.

FIG. 11 depicts the structure of 3D-(6-acyl) PHAD (or 3,6-acyl PHAD).

### DETAILED DESCRIPTION

### Definitions

“About” and “approximately” shall generally mean within an acceptable degree of error for the quantity measured given the nature or precision of the measurements. Typically, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. “Colloid” as used herein, refers to any liquid or solid composition comprising multimolecular aggregate microstructures having diameters or lengths on the scale of 1 nm to 10 um. Such microstructures include but are not limited to micelles, liposomes, vesicles, nanoparticles, microparticles, etc. The microstructures may be spherical, oval, oblong, flat, or any other shape.

“Micelles,” as used herein, is an art-recognized term and refers to particles of colloidal dimensions that exist in equilibrium with the molecules or ions in solution from which it is formed. It is an aggregate (or supramolecular assembly) of molecules dispersed in a liquid, forming a colloidal suspension (also known as associated colloidal system). A typical micelle in water forms an aggregate with the hydrophilic “head” regions in contact with surrounding solvent, sequestering the hydrophobic single-tail regions in the micelle center.

“Liposome,” as used herein, is an art-recognized term and refers to a spherical vesicle having at least one lipid bilayer. Liposomes can be prepared by disrupting biological membranes (such as by sonication).

“Vesicle,” as used herein is an art-recognized term and refers to a membranous fluid filled sac surround by a lipid bilayer.

“Nanoparticle,” as used herein, is an art-recognized term and is typically defined as a particle of matter that is between 1 and 100 nanometers (nm) in diameter. The term is sometimes used for larger particles, up to 500 nm.

“Microparticle,” as used herein, is an art-recognized term and is defined to be particles between 1 and 1000 μm in size.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic (i.e., it is intended to diminish, ameliorate, or stabilize the existing unwanted condition or side effects thereof). Treating a respiratory viral infection may include: alleviation or elimination of symptoms such as runny nose, sneezing, itchy watery eyes, cough, fatigue, headache, sore throat, or congestion.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

A “patient,” “subject,” or “individual” are used interchangeably and refer to either a human or a non-human animal. These terms include mammals, such as humans, non-human primates, livestock animals (including bovines, porcines, etc.), companion animals (e.g., canines, felines, etc.) and rodents (e.g., mice and rats). In some embodiments, the subject is a human.

The phrase “pharmaceutically acceptable excipient” as used herein means a pharmaceutically acceptable material, composition or vehicle, such as a liquid or solid filler, diluent, lubricant, binder, carrier, humectant, disintegrant, solvent or encapsulating material, that one skilled in the art would consider suitable for rendering a pharmaceutical formulation suitable for administration to a subject. Each excipient must be “acceptable” in the sense of being compatible with the other ingredients of the formulation, as well as “pharmaceutically acceptable” as defined above. Examples of materials which can serve as pharmaceutically acceptable excipients include but are not limited to: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; silica, waxes; oils, such as corn oil and sesame oil; glycols, such as propylene glycol and glycerin; polyols, such as sorbitol, mannitol and polyethylene glycol; esters, such as ethyl oleate and ethyl laurate; agar; buffering agents; alginic acid; pyrogen-free water; isotonic saline; Ringer's solution; and other non-toxic compatible substances routinely employed in pharmaceutical formulations.

A “therapeutically effective amount” or a “therapeutically effective dose” of a drug or agent is an amount of a drug or an agent that, when administered to a subject, will have the intended therapeutic effect. The full therapeutic effect does not necessarily occur by administration of one dose and may occur only after administration of a series of doses. Thus, a therapeutically effective amount may be administered in one or more administrations. The precise effective amount needed for a subject will depend upon, for example, the subject's size, health and age, and the nature and extent of the condition being treated.

### Allergies and the Innate Immune System

The complex pathways leading to T-helper cell type 2 (Th2) immune responses against food antigens cannot be attributed to a single driving force.

While not being bound by theory, it is believed that the methods of adjuvant therapy for AIT disclosed herein produce certain protective cytokines, including interferons (IFNs) and interleukins (ILs). MPLA upregulates protective cytokines via interaction with Toll-like receptor 4 (TLR4), which upon stimulation activates 2 signal transduction pathways: one mediated by myeloid differentiation factor 88 (MyD88); and another mediated by toll-IL-1 receptor domain containing adaptor inducing IFN-0 (TRIF). MPLA upregulates specific cytokines, including IL-6, IL-10, IFN-7 (Type II IFN), Type I IFNs (IFN-α and IFN-0), IP-10, IL-17, and IL-8 which are secreted from specific immune cells. Specifically, IL-10 has a well-defined role as an inhibitor of pro-inflammatory cytokine expression, such as IL-1β, IL-6, and TNF-α (Saraiva M, O'Garra A. The regulation of IL-10 production by immune cells. Nat Rev Immunol. 2010; 10(3):170-181. doi:10.1038/nri2711 and Margarida Saraiva, Paulo Vieira, Anne O'Garra; Biology and therapeutic potential of interleukin-10. J Exp Med 6 Jan. 2020; 217 (1): e20190418. doi: https://doi.org/10.1084/jem.20190418). IL-10 is key to the induction of tolerance in food allergies through the regulatory activity of FoxP3+ and Tr T cells (Chen T K, Lee J H, Yu H H, Yang Y H, Wang L C, Lin Y T, et al. Association between human IL-10 gene polymorphisms and serum IL-10 level in patients with food allergy. J Formos Med Assoc. Elsevier Taiwan LLC; 2012; 111(12):686-92, Syed A, Garcia M A, Lyu S C, et al. Peanut oral immunotherapy results in increased antigen-induced regulatory T-cell function and hypomethylation of forkhead box protein 3 (FOXP3). J Allergy Clin Immunol. 2014; 133(2):500-510. doi:10.1016/j.jaci.2013.12.1037). This regulatory activity includes inhibition of the development of allergen-specific Th2 and T helper type 1 (Th1) cell responses and the direct or indirect suppression of effector cells of allergic inflammation.

In addition, the upregulation of Type I IFNs have demonstrated specific antibacterial capability against gram negative bacteria. Although IFNs have been used in previous treatments or combination treatments, the timing and method of administration of IFN is vital to its effectiveness against infection. In contrast to exogenous interferon, compositions comprising an MPLA compound in accordance with the invention offer a number of advantages, including the elicitation of the innate immune response, notably, the increased expression of endogenous Type I interferons.

Synthetic and bacterially-derived MPLA compounds are recognized by toll-like receptor 4 (TLR4). Interaction with TLR4 stimulates the production of protective cytokines including interferons (IFNs) (e.g., IFN-α, IFN-γ), IP-10, IL-8, IL-17, and TNF-α, or combinations thereof. Interferons are a type of cytokine associated with the innate immune system and are released by sentinel cells (e.g., macrophages, dendritic cells) when TLRs on sentinel cells come in contact with PAMPs. Interferons interfere with bacteremia, increase bacterial clearance, and are the body's first line of defense against bacterial infections, binding to cell surface receptors to activate the transcription of hundreds of genes.

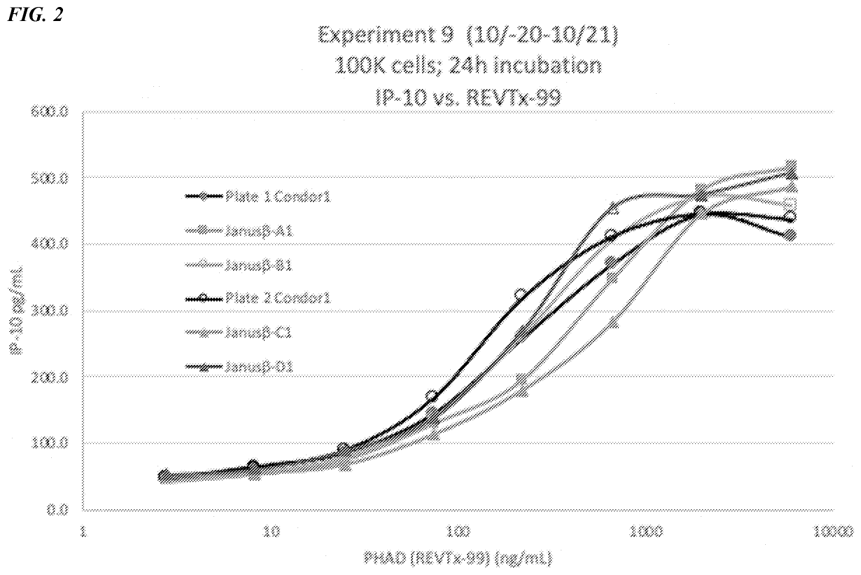

Robust upregulation of IP-10, as a result of TLR4 stimulation by MPLA compounds, has been demonstrated in vitro in mouse macrophages (FIG. 2). Interferon-β is required for production of IP-10, therefore IP-10 is considered a surrogate marker for Type I interferon activity.

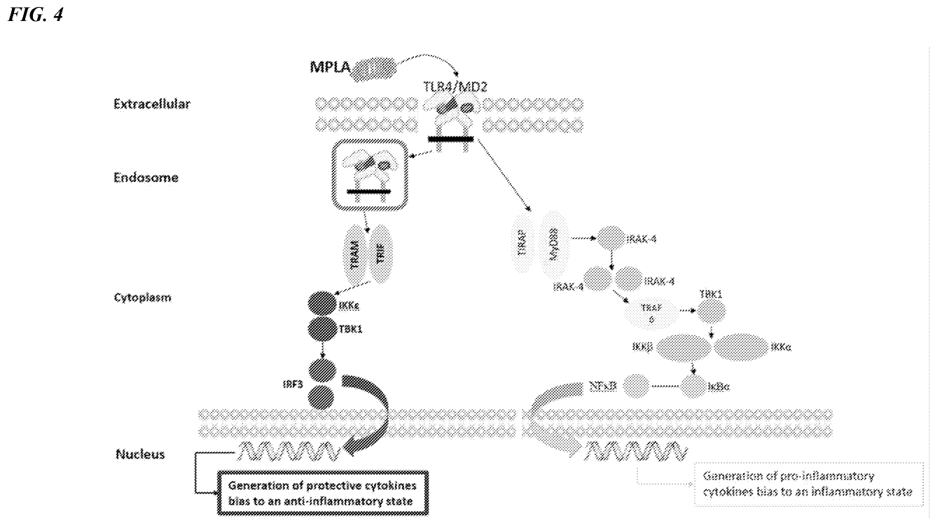

The stimulation of TLR4 in response to MPLA compounds leads to generation of IL-10 and Type I interferons preferentially through the TRIF pathway (FIG. 4). A negative reciprocal feedback loop has been observed to exist between IL-10 and Type I interferon activity and Th2-biased cellular activity (Gonzalez-van Horn 2015). This relationship is illustrated in FIG. 6, which details IL-10 and Type I interferon production blocking activation of Th2 cellular activity at the molecular level (suppression of GATA3 expression, preventing access to transcription mediated expression for H3K27me3 and Total H3), which prevents generation of Th2 cytokines (IL-4, IL-5, and IL-13), reducing or eliminating secretion of IgE, therefore reducing or preventing allergic symptoms. It has been demonstrated that Type I interferons reduce or prevent Th2-biased cellular activity, which facilitates the allergic response, destabilizing establishment of Th2-biased cellular activity.

Also while not being bound by theory, it is believed that the present methods are capable of reducing eotaxin activation through indirect competition for the native receptor, CCR3, as IP-10 is capable of binding to CCR3. In addition to this activity, the present methods are further capable of reducing allergic responses via TLR4 mediated recruitment of exosomes from stimulated epithelial cells. In the anterior portion of the nose, these exosomes release antimicrobial peptides and nitric oxide into nasal mucus that can destroy foreign invaders.

The invention provides a safe and effective method of augmenting the treatment of allergic disease in subjects indicated to receive AIT treatment by acting as a co-therapy with the respective form of AIT. As used herein, “AIT” refers to any treatment prescribed to prevent or manage symptoms of allergic reactions by exposing subjects to small increasing doses of an allergen. Typically, the subject is a human.

In particular embodiments, the allergen is a food allergen.

The method of treatment includes administration of a monophosphoryl Lipid A, or MPLA, compound. The MPLA compound may be isolated from gram-negative bacterial cell walls or may be a synthetically prepared MPLA compound.

### MPLA Compounds

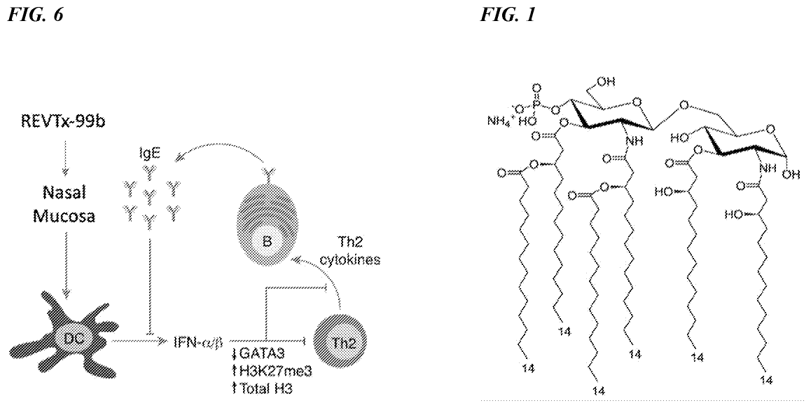

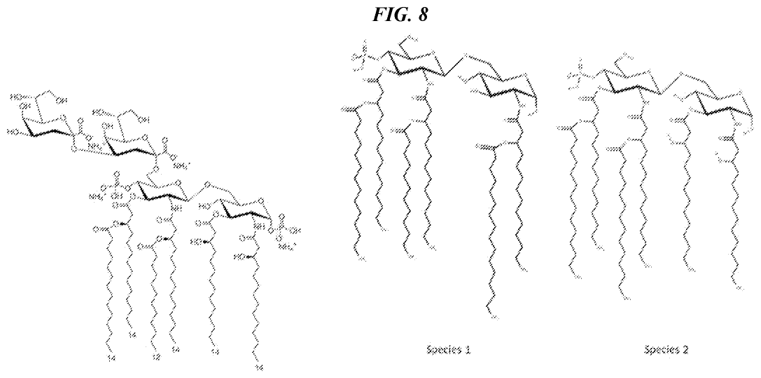

The pharmaceutical compositions of the present disclosure comprise a monophosphoryl lipid A (MPLA) like compound. MPLA was originally isolated from lipopolysaccharide obtained from gram-negative bacterial cell walls (FIG. 8). Bacterially-derived MPLA is typically a mixture of several different species. FIG. 1 shows one of the predominant species of bacterially-derived MPLA. As an example, MPLA may be derived from Salmonella minnesota R595 lipopolysaccharides. As will be understood, MPLA may also be derived from other Salmonella species. The bacterial LPS may be processed via sequential acid and alkaline hydrolysis steps to remove polysaccharide side chains, phosphate groups, and to partially remove a portion of the acetyl side groups. The crude MPLA may then be purified. The final MPLA product is a mixture of heptaacyl-, hexaacyl-, and pentaacyl-monophosphorylated glucosamine disaccharide linked β1. Diaacetyl, triaacetyl, and tetraacetyl, if present, are considered impurities. The acylated lipids vary and include lauroyl, myristoyl, and palmitoyl. While the relative ratio of each species can vary from batch to batch, the predominant species produced are the hexaacylated disaccharide products.

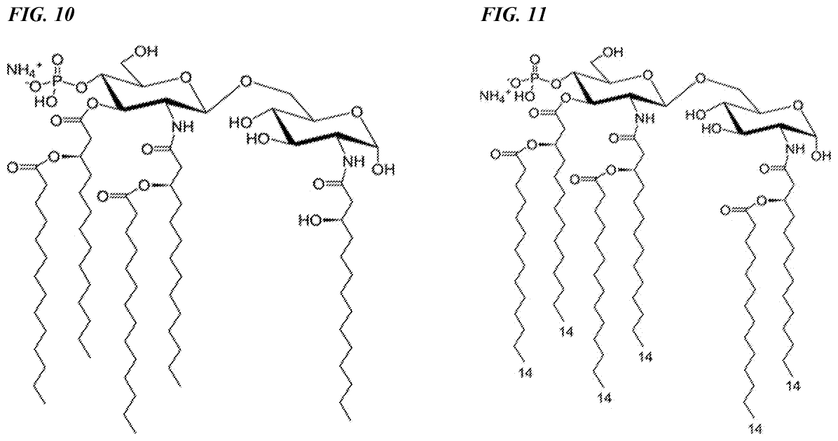

The major species found in bacterially-derived MPLA have been chemically synthesized and have comparable immunostimulatory properties to the bacterially-derived material. Examples of synthetic MPLA compounds suitable for use in the present invention include phosphorylated hexaacyl disaccharide (PHAD®) (also known as glucopyranosyl lipid A, or GLA) (FIG. 1), 3D-PHAD (or 3-acyl-PHAD) (also known as monophosphoryl 3-deacyl lipid A) (FIG. 10), and 3D-(6-acyl) PHAD (or 3,6-acyl PHAD) (FIG. 11). Synthetic variations of MPLA that are also suitable and within the scope of the invention include those wherein the fatty acid chain length varies between 10-20 carbons and those wherein the degree of acylation is penta-, hexa-, or hepta-.

PHAD is chemically equivalent to a major component of bacterially-derived MPLA. PHAD is also similar to bacterially-derived MPLA in biologic effect.

The MPLA compound administered in accordance with the present invention stimulates the innate immune system to produce certain protective anti-inflammatory cytokines, including interferons (IFNs) and interleukins (ILs). MPLA upregulates the production of anti-inflammatory cytokines via agonistic interferon activity with TLR4. While not wishing to be bound by theory, it is believed that the mechanism of the present invention is attributable to its ligand activity with TLR4. The MPLA compound administered in accordance with the present invention may contribute to mechanical barrier functions in the nose by acting as a soap-like agent, thereby, breaking apart cellular components of the antigen, rendering the remaining artifacts of the antigen inert. The MPLA compound administered in accordance with the present invention may provoke the release of exosomes to release antimicrobial components (as described earlier). The MPLA compound administered in accordance with the present invention may mimic the immune induction activities of rough or smooth LPS (as described earlier).

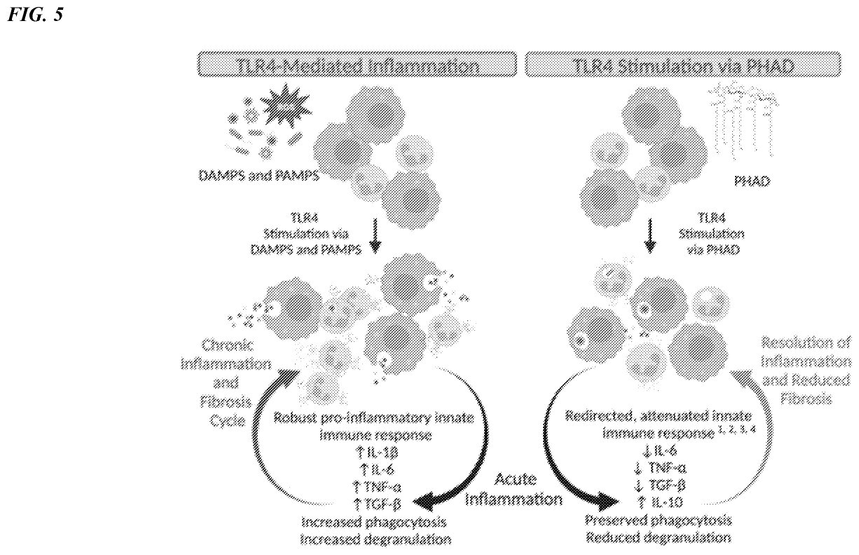

The present invention may have a preference for the TRIF signaling pathway [FIGS. 4 and 5]) that leads to the synthesis of IL-10 and Type I interferons, the principal Th1 effector cytokines that induce macrophage/monocyte activation, mast cell engagement, and dendritic cell maturation.

The present invention is an intravenous, oral or sublingual, subcutaneous, intramuscular, or intranasal MPLA composition that may present properties similar to both rough and smooth LPS. Intravenous, oral or sublingual, subcutaneous, intrathecal, or intramuscular administration of a TLR4 ligand may advantageously ensure systemic exposure to multiple organs, and induce the innate immune response (nose, mouth, intestines, and other mucosal sites). Alternatively, intranasal administration of a TLR4 ligand is a useful deposition site due to the high density of myeloid antigen-presenting cells with expression of TLR4 in the nasal cavity. Activation of TLR4 in nasal mucosa cells generates an advantage for NALT to be the induction site of common mucosal immunity, stimulating both cellular and humoral responses to effector sites (mouth, intestines, and other mucosal surfaces).

### Compositions

In some embodiments, the MPLA compound is administered with one or more pharmaceutically acceptable excipients. For example, the MPLA compound can be formulated for intranasal delivery as a dry powder, as an aqueous solution, an aqueous suspension, a colloid, a water-in-oil emulsion, or as a liposomal formulation. In certain embodiments, the spray-dried powder is reconstituted with water prior to administration. Useful excipients for intranasal formulation include but are not limited to trehalose, cyclodextrin, mannitol, charged and uncharged lipids, such as phospholipids including dipalmitoyl phosphatidyl choline (DPPC), dioleoylphosphatidylcholine (DOPC), and cholesterol.

In certain embodiments, formulations of MPLA may contain an organic solvent, pH modifiers, pH buffers, tonicity modifiers, bulking agents, stabilizers, preservatives, detergents, mucoadhesives, or secondary immunostimulatory agents. Secondary immunostimulatory agents include gonadocorticoids, deoxycholic acid, vitamin D, and beta-glucans, among others. Suitable buffers include sodium chloride-based or potassium chloride-based solutions such as phosphate buffered saline, potassium buffered saline, or borate buffered saline. In some embodiments, the buffer may contain salts, detergents, or carbohydrates which preserve the MPLA upon drying and aid in resolubilizing the MPLA upon encounter with a liquid. Suitable carbohydrates include trehalose, cyclodextrin, sucrose, glucose, and mannose (or its reduced form, mannitol). Suitable organic solvents include ethanol, t-butanol, and methanol. Suitable mucoadhesives include glycosylaminoglycans (GAGS) including chondroitin sulfate, chitosan, and hyaluronic acid. In certain embodiments, the formulation may contain ionic or nonionic surfactants such as poloxamer 407 and poloxamer 181. In certain embodiments, the organic solvent may comprise up to 15% of the total end volume of the administered drug product solution. In preferred embodiments, the organic solvent may comprise up to 5% of the total end volume of the administered drug product solution.

In some embodiments, the MPLA is formulated at a pH between 4 and 9. In certain preferred embodiments, the pH is between 5 and 8 and most preferably between 6.5 and 7.5.

In some embodiments, the MPLA is formulated at an osmolality between 100 and 700 mOsmol/kg. In certain preferred embodiments, the osmolality is between 290 and 500 mOsmol/kg.

### Dosage Forms and Administration

MPLA compounds can be administered by several different routes. The choice of the route is dependent on multiple factors including the need (or not) for systemic exposure, the desire for the MPLA compound to reach a particular organ quickly, patient tolerability, and compliance.

Methods of systemic delivery include those methods known in the art that provide delivery of the active molecule (e.g., the drug) to the circulatory system with distribution throughout the body. Systemic delivery methods include intramuscular, intravenous, subcutaneous, intraperitoneal, sublingual, and oral. As will be understood, any method of systemic delivery is suitable for use with the invention. Particularly suitable methods of systemic delivery include oral, intramuscular, and intravenous delivery.

In some embodiments, it may be desirable to have the drug interact with only the mucosal tissue, thereby providing no or minimal systemic exposure. Methods for mucosal delivery include those methods known in the art that provide delivery of the active molecule to mucous membranes. Mucosal delivery methods include intranasal, intrabuccal, sublingual, and oral. Particularly suitable methods for mucosal delivery include intranasal delivery.

In these embodiments, the composition comprising the MPLA compound may be formulated to be delivered to the nasal passages or nasal vestibule of the subject as droplets, an aerosol, micelles in solution, lipid or liquid nanospheres, liposomes, lipid or liquid microspheres, a solution spray, or a powder. The composition can be administered by direct application to the nasal passages or may be atomized or nebulized for inhalation through the nose or mouth.

In some embodiments, the method comprises administering a nasal spray, medicated nasal swab, medicated wipe, nasal drops, or aerosol to the subject's nasal passages or nasal vestibule. To this end, viscosity modifying agents that may be deployed to optimize the product for the application format may include cetyl alcohol, stearyl alcohol, carnauba wax, stearic acid, xanthan gum, magnesium aluminum silicate, gelatin, carbomer, poloxamers, PEGs, waxes, starches, castor oil derivatives, fatty acids, fatty alcohols, and lecithin.

In some embodiments, the compositions of the present invention can be delivered using a small needle-free nasal spray device, which can allow (self) administration with little or no prior training to deliver a desired dose. The apparatus can comprise a reservoir containing a quantity of the composition. The apparatus may comprise a pump spray for delivering one or more metered doses to the nasal cavity of a subject. The device may advantageously be single-dose use or multi-dose use. It further may be designed to administer the intended dose with multiple sprays, e.g., two sprays, e.g., one in each nostril, or as a single spray, e.g., in one nostril, or to vary the dose in accordance with the body weight or maturity of the patient. In some embodiments, nasal drops may be prepacked in pouches or ampoules that may be opened immediately prior to use and squeezed or squirted into the nasal passages. In some embodiments, the nasal spray or drops may be accomplished by time of use reconstitution of the product powder with an aqueous vehicle immediately prior to administration. In some embodiments, the nasal spray or drops may be accomplished by reconstitution of the solid drug product powder contained in a suitable delivery device using an aqueous vehicle in some time period in which the drug product is deemed stable in solution format prior to patient administration.

In certain embodiments, the compositions are suitable for parenteral administration to a mammal, most preferably by injection or intravenous infusion, and in some embodiments the compositions may comprise one or more pharmaceutically acceptable excipients. Suitable excipients include pharmaceutically acceptable buffers, stabilizers, local anesthetics, and the like. The composition may be adapted for direct injection or intravenous infusion, or for addition to an intravenous drip solution for gradual infusion, through appropriate use of excipients and packaging and delivery means well-known in the art.

In other embodiments, the invention provides a pharmaceutical package, comprising a vial or ampoule containing an MPLA compound in the form of a reconstitutable powder or a solution suitable for injection or infusion, together with instructions for administering the composition to a patient in need thereof. Instructions include but are not limited to written and/or pictorial descriptions of: the active ingredient, directions for diluting the composition to a concentration suitable for administration, suitable indications, suitable dosage regimens, contraindications, drug interactions, and any adverse side-effects noted in the course of clinical trials.

In alternative embodiments, the pharmaceutical package may comprise a plastic bag containing from 20 ml to 2 L of a pharmaceutical composition of the invention, in the form of a solution suitable for intravenous administration, together with instructions as described above.

In alternative embodiments, a pharmaceutical composition of the invention may be in a form adapted for oral dosage, such as for example a syrup or palatable solution; a form adapted for topical application, such as for example a cream or ointment; or a form adapted for administration by inhalation, such as for example a microcrystalline powder or a solution suitable for nebulization. Methods and means for formulating pharmaceutical ingredients for alternative routes of administration are well-known in the art, and it is to be expected that those skilled in the relevant arts can adapt these known methods to the MPLA compounds and formulations described in the present invention.

The present invention provides pharmaceutically acceptable compositions comprising a therapeutically effective amount of one or more MPLA compounds, formulated together with one or more pharmaceutically acceptable excipients. The pharmaceutical compositions of the present invention may be formulated for administration in solid or liquid form, including forms adapted for oral administration, for example, aqueous or non-aqueous solutions or suspensions, tablets, powders, and granules; administration by inhalation, for example, aerosols, solutions for nebulization, or dry powders; parenteral administration, for example sterile solutions or suspensions; topical application, for example lotions, creams, ointments or sprays; ophthalmic administration; or intravaginal or intrarectal administration, for example pessaries, suppositories, creams or foams. Preferably, the pharmaceutical preparation is adapted for parenteral administration, more preferably it is a non-pyrogenic solution adapted for intravenous administration.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention may optionally be scored or prepared with coatings and shells, such as enteric coatings and other coatings well known in the pharmaceutical-formulating art. They may also be formulated so as to provide slow or controlled release of the modified therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally in a delayed manner. Examples of embedding compositions that can be used include polymeric substances and waxes. The MPLA compound can also be in micro-encapsulated form, if appropriate, with one or more of the above-described excipients.

Liquid dosage forms for oral administration of MPLA compounds include pharmaceutically acceptable emulsions, microemulsions, solutions, suspensions, syrups, and elixirs. In addition to the MPLA compound, the liquid dosage forms may contain inert diluents commonly used in the art, such as, for example, water or other solvents, solubilizing agents, and emulsifiers.

Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

In dry powder formulations adapted for inhalation, the particle size of the particulate medicament should be such as to permit inhalation of substantially all of the medicament into the lungs upon administration of the aerosol formulation and will thus desirably be less than 20 microns, preferably in the range 1 to 10 microns, more preferably 1 to 5 microns. The particle size of the medicament may be reduced by conventional means, for example by milling or micronization. The aerosol formulation preferably contains 0.5-30% w/w of an MPLA compound relative to the total weight of the formulation.

The propellant may optionally contain an adjuvant having a higher polarity and/or a higher boiling point than the propellant. Polar adjuvants which may be used include (e.g. C2-6) aliphatic alcohols and polyols such as ethanol, isopropanol and propylene glycol, preferably ethanol. In general, only small quantities of polar adjuvants (e.g. 0.05-3.0% w/w) may be required to improve the stability of the dispersion. However, the formulations of the invention are preferably substantially free of polar adjuvants, especially ethanol. Suitable propellants include trichlorofluoromethane (propellant 11), dichlorodifluoromethane (propellant 12), dichlorotetrafluoroethane (propellant 114), tetrafluoroethane (propellant 134a) and 1,1-difluoroethane (propellant 152a), saturated hydrocarbons such as propane, n-butane, isobutane, pentane and isopentane, and alkyl ethers such as dimethyl ether. In general, up to 50% w/w of the propellant may comprise a volatile adjuvant, for example 1 to 30% w/w of a volatile saturated C1-C6 hydrocarbon.

The aerosol formulations according to the invention may optionally comprise one or more surfactants that are physiologically acceptable upon administration by inhalation.

For administration by inhalation, the drug is suitably inhaled from a nebulizer, from a pressurized metered dose inhaler or as a dry powder from a dry powder inhaler optionally using gelatin, plastic or other capsules, cartridges, blister packs and/or strips.

For use in dry powder inhalers, the active ingredient can be modified by spray drying or compression to form a powder with suitable flow properties. More commonly a diluent or carrier is added which is generally non-toxic and inert to the medicament. Examples of such carriers are polysaccharides e.g. starch and cellulose, dextran, lactose, glucose, mannitol, and trehalose. The carrier can be further modified by the addition of surface modifiers, pretreatment to form low rugosity particles, addition of glidants, and flavor masking or modifying agents.

Pharmaceutical compositions of this invention suitable for parenteral administration comprise an MPLA compound in combination with one or more pharmaceutically acceptable sterile isotonic aqueous or non-aqueous solutions, or sterile powders which may be reconstituted into sterile injectable solutions or dispersions just prior to use, which may contain antioxidants, buffers, bacteriostats, or solutes which render the formulation isotonic with the blood of the intended recipient or suspending or thickening agents.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions.

Examples of pharmaceutically acceptable antioxidants include but are not limited to ascorbic acid, cysteine hydrochloride, sodium metabisulfite, sodium sulfite, ascorbyl palmitate, butylated hydroxyanisole (BHA), butylated hydroxytoluene (BHT), propyl gallate, alpha-tocopherol, and chelating agents such as citric acid, ethylenediamine tetraacetic acid (EDTA), sorbitol, tartaric acid, phosphoric acid, and the like.

Injectable depot forms are made by forming microencapsuled matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes, vesicles, or microemulsions that are compatible with body tissue.

Dosage forms for the topical or transdermal administration of a compound of this invention include powders, sprays, ointments, pastes, creams, lotions, gels, solutions, patches and inhalants. The MPLA compound may be mixed under sterile conditions with a pharmaceutically acceptable carrier, and with any preservatives, buffers, or propellants that may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Ophthalmic formulations, eye ointments, powders, solutions and the like, are also contemplated as being within the scope of this invention.

Formulations of the present invention which are suitable for vaginal administration include pessaries, tampons, creams, gels, pastes, foams or spray formulations, containing such carriers as are known in the art to be appropriate. Such formulations may be prepared, for example, by mixing one or more MPLA compounds with one or more suitable nonirritating excipients comprising, for example, cocoa butter, polyethylene glycol, or a suppository wax, which is solid at room temperature but liquid at body temperature and, therefore, will melt in the rectum or vaginal cavity and release the MPLA compound.

### Therapeutic Doses and Regimens

In certain embodiments, an MPLA compound is administered in a total dose between about 0.001 to about 10 milligrams. In other embodiments, the total dose is about 1 to 10,000 micrograms. In preferred embodiments, the total dose administered is between 1.0 and 1000 micrograms. In particularly preferred embodiments, the total dose is between 10 to 500 micrograms. In certain embodiments, MPLA is administered in a total dose between 50 to 200 micrograms. In some embodiments, the total dose given may be administered intranasally, and may be divided in equal or unequal parts between both nostrils. In preferred embodiments, the total dose administered is between about 1.0 and 500 micrograms. In other embodiments, the total dose administered is between about 10 and 100 micrograms, 50 to 100 micrograms, 50 to 1000 micrograms, 100 to 500 micrograms, or 50 to 200 micrograms. In particularly preferred embodiments, the total dose is about 10 to 200 micrograms.

In other embodiments of the invention, an MPLA compound is given as a single dose. In other embodiments, MPLA compound is given multiple times. In the case of multiple doses, MPLA compounds may be given daily, bi-weekly, weekly, or monthly. The exact frequency of dosing and the dose required at each interval will depend on multiple factors including the type of allergic condition being treated, the rate of allergic relapse, and patient tolerability. Early in the treatment phase, the dose and/or dosing frequency may be greater with a gradual decrease in both dose and/or dosing frequency as the allergic response diminishes to maintenance of a steady state (lack of allergic response)

In some embodiments of the invention, the MPLA compound is administered prior to beginning AIT, during AIT, or after the completion of AIT.

The total dose given may be administered intravenously, and may be administered as a bolus or as an infusion. The total dose given may be administered orally or sublingually, subcutaneously, or intramuscularly.

### Compositions

In certain embodiments, the pharmaceutical composition is an aqueous composition. In some such embodiments, the pharmaceutical compositions may contain an organic solvent. In certain embodiments, the organic solvent may comprise up to 15% of the total end volume of the administered drug product solution. In preferred embodiments, the organic solvent may comprise up to 5% of the total end volume of the administered drug product solution.

In certain embodiments, the organic solvent is miscible with water, such as an organic solvent selected from an alcohol, glycerin, low molecular weight polyethylene glycol, and low molecular weight poloxamers. In certain preferred embodiments, the organic solvent is an alcohol, e.g., methanol, ethanol, isopropanol, t-butanol, or preferably ethanol.

In certain embodiments, the composition further comprises a sugar. In some embodiments, the sugar is chosen from a monosaccharide, a disaccharide, a trisaccharide, a linear oligosaccharide, a branched oligosaccharide, a cyclic oligosaccharide, a linear polysaccharide, a branched polysaccharide, or any combination thereof.

In some embodiments, the monosaccharide is selected from glucose, dextrose, fructose, galactose, xylose, ribose, and any combination thereof.

In some embodiments, the disaccharide is selected from trehalose, sucrose, maltose, lactose, and any combination thereof.

In some embodiments, the trisaccharide is selected from nigerotriose, maltotriose, melezitose, maltotriulose, raffinose, kestose, and any combination thereof.

In some embodiments, the linear or branched oligosaccharide is selected from nigerotetraose, maltotetraose, lychnose, nystose, sesamose, stachyose.

In some embodiments, the cyclic oligosaccharide is selected from alpha-cyclodextrin, beta-cyclodextrin, or gamma-cyclodextrin.

In some embodiments, the linear or branched polysaccharide is selected from starch, glucan, chitosan, pectin, carboxymethyl cellulose, glycosylaminoglycans, hyaluronic acid, cellulose derivatives, hydroxypropylmethylcellulose (HPMC), dextran, and any combination thereof.

In some preferred embodiments, the sugar comprises trehalose, beta-cyclodextrin, or both.

In certain embodiments, the pharmaceutical compositions further comprise one or more surfactants. In certain embodiments, the one or more surfactants are selected from carboxymethyl cellulose, dodecyltrimethylammonium bromide (DTAB), n-dodecyl octa(ethylene oxide) (C12E8), n-dodecyl tetra (ethylene oxide) (C12E4), dioctanoyl phosphatidylcholine (C8-lecithin), Polyoxyl 35 castor oil, Cremophor EL (CrEL), Octaethylene glycol monododecyl ether (C12E8), hexadecyltrimethylammonium bromide (CTAB), polypropylene oxide (PPO), polyethylene oxide (PEO), PEO-poly(D,L-lactic acid-co-caprolactone) (PEO-PDLLA), sodium dodecyl sulfate (SDS), triethylamine, trimethylamine and any combination thereof. In some embodiments, the pharmaceutical compositions further comprise one or more surfactants. In certain embodiments, the one or more surfactants are selected from carboxymethyl cellulose, dodecyltrimethylammonium bromide (DTAB), n-dodecyl octa(ethylene oxide) (C12E8), n-dodecyl tetra (ethylene oxide) (C12E4), dioctanoyl phosphatidylcholine (C8-lecithin), Polyoxyl 35 castor oil, Cremophor EL (CrEL), Octaethylene glycol monododecyl ether (C12E8), hexadecyltrimethylammonium bromide (CTAB), polypropylene oxide (PPO), polyethylene oxide (PEO), PEO-poly(D,L-lactic acid-co-caprolactone) (PEO-PDLLA), sodium dodecyl sulfate (SDS), and any combination thereof. In certain preferred embodiments, the one or more surfactants is carboxymethyl cellulose.

In certain embodiments, the pharmaceutical compositions may contain excipients, additives, bulking agents, and mucoadhesive agents. These may include mannitol, trehalose, dextrose, cyclodextrin, and hydroxypropyl methylcellulose (HPMC). In preferred embodiments, the excipients HP-β-Cyclodextrin and dextrose are employed.

In certain embodiments, the pharmaceutical compositions further comprise a phospholipid selected from phosphatidic acid (PA), phosphatidylcholine (PC), phosphatidylglycerol (PG), phophatidylethanolamine (PE), phophatidylinositol (PI), phosphatidylserine (PS), sphingomyelin (including brain sphingomyelin), lecithin, lysolecithin, lysophosphatidylethanolamine, cerebrosides, diarachidoylphosphatidylcholine (DAPC), didecanoyl-L-alpha-phosphatidylcholine (DDPC), dielaidoylphosphatidylcholine (DEPC), dilauroylphosphatidylcholine (DLPC), dilinoleoylphosphatidylcholine, dimyristoylphosphatidylcholine (DMPC), dioleoylphosphatidylcholine (DOPC), dipalmitoylphosphatidylcholine (DPPC), distearoylphosphatidylcholine (DSPC), 1-palmitoyl-2-oleoyl-phosphatidylcholine (POPC), diarachidoylphosphatidylglycerol (DAPG), didecanoyl-L-alpha-phosphatidylglycerol (DDPG), dielaidoylphosphatidylglycerol (DEPG), dilauroylphosphatidylglycerol (DLPG), dilinoleoylphosphatidylglycerol, dimyristoylphosphatidylglycerol (DMPG), dioleoylphosphatidylglycerol (DOPG), dipalmitoylphosphatidylglycerol (DPPG), distearoylphosphatidylglycerol (DSPG), 1-palmitoyl-2-oleoyl-phosphatidylglycerol (POPG), diarachidoylphosphatidylethanolamine (DAPE), didecanoyl-L-alpha-phosphatidylethanolamine (DDPE), dielaidoylphosphatidylethanolamine (DEPE), dilauroylphosphatidylethanolamine (DLPE), dilinoleoylphosphatidylethanolamine, dimyristoylphosphatidylethanolamine (DMPE), dioleoylphosphatidylethanolamine (DOPE), dipalmitoylphosphatidylethanolamine (DPPE), distearoylphosphatidylethanolamine (DSPE), 1-palmitoyl-2-oleoyl-phosphatidylethanolamine (POPE), diarachidoylphosphatidylinositol (DAPI), didecanoyl-L-alpha-phosphatidylinositol (DDPI), dielaidoylphosphatidylinositol (DEPI), dilauroylphosphatidylinositol (DLPI), dilinoleoylphosphatidylinositol, dimyristoylphosphatidylinositol (DMPI), dioleoylphosphatidylinositol (DOPI), dipalmitoylphosphatidylinositol (DPPI), distearoylphosphatidylinositol (DSPI), 1-palmitoyl-2-oleoyl-phosphatidylinositol (POPI), diarachidoylphosphatidylserine (DAPS), di decanoyl-L-alpha-phosphatidylserine (DDPS), dielaidoylphosphatidylserine (DEPS), dilauroylphosphatidylserine (DLPS), dilinoleoylphosphatidylserine, dimyristoylphosphatidylserine (DMPS), dioleoylphosphatidylserine (DOPS), dipalmitoylphosphatidylserine (DPPS), distearoylphosphatidylserine (DSPS), 1-palmitoyl-2-oleoyl-phosphatidylserine (POPS), diarachidoyl sphingomyelin, didecanoyl sphingomyelin, dielaidoyl sphingomyelin, dilauroyl sphingomyelin, dilinolcoyl sphingomyelin, dimyristoyl sphingomyelin, sphingomyelin, dioleoyl sphingomyelin, dipalmitoyl sphingomyelin, distearoyl sphingomyelin, 1-palmitoyl-2-oleoyl-sphingomyelin, and any combination thereof.

In certain embodiments, the pharmaceutical composition is free or substantially free of salt (e.g., NaCl).

In certain embodiments, the pharmaceutical composition has an MPLA concentration of about 0.01 μg/mL to about 1,000 μg/mL. In other embodiments, the pharmaceutical composition has an MPLA concentration of about 1 μg/mL to about 10,000 μg/mL, about 1 μg/mL to about 30 μg/mL, about 50 μg/mL to about 500 μg/mL, about 50 μg/mL to about 200 μg/mL, about 0.20 μg/mL to about 20 μg/mL, or about 25 μg/mL to about 50 μg/mL. In certain embodiments, the pharmaceutical composition has an MPLA concentration of about 125 μg/mL or about 250 μg/mL. In other embodiments, the pharmaceutical composition has an MPLA concentration of about 2.5 μg/mL. In certain embodiments, the pharmaceutical composition has an MPLA concentration of about 1.25 μg/mL.

In certain embodiments, the pharmaceutical compositions further comprise a stabilizer or multiple stabilizers. In some embodiments, the stabilizer comprises trehalose. In other embodiments, the stabilizer comprises HP-β-Cyclodextrin and trehalose. In certain preferred embodiments, the stabilizer is dextrose. In other preferred embodiments, the stabilizer is HP-β-Cyclodextrin. In some most preferred embodiments, both HP-β-Cyclodextrin and dextrose are used as stabilizers.

In certain embodiments, the pharmaceutical composition is a dry powder.

In certain embodiments, the colloidal or micellar solution comprises particles having an average diameter of about 1 nm to about 1000 nm. In certain preferred embodiments, the particles have an average diameter of about 50 nm to about 500 nm. In other more preferred embodiments, the particles have an average diameter of about 100 nm to about 500 nm. In other embodiments, the particles have an average diameter of about 500 nm to about 1000 nm. In certain embodiments, the particles have an average diameter of about 10 nm to about 200 nm. In preferred embodiments, the particles have an average diameter of about 10 nm to about 100 nm. In certain embodiments, the particles have an average diameter of less than 50 nm. In certain embodiments, the particles have an average diameter of less than 100 nm. In certain embodiments, the particles have an average diameter of less than 150 nm. In certain embodiments, the particles have an average diameter of less than 200 nm. In certain embodiments, the particles have an average diameter of less than 250 nm. In certain embodiments, the particles have an average diameter of less than 500 nm.

In certain embodiments, the pharmaceutical compositions further comprise a mucoadhesive agent. In certain embodiments, the mucoadhesive agent is selected from cellulose derivatives, polyacrylates, a starch, chitosan, glycosylaminoglycans, hyaluronic acid, cellulose derivatives, polyacrylates, and any combination thereof.

In certain preferred embodiments, the pharmaceutical compositions further comprise a pH modifier, an emulsifier, a pH buffer, a tonicity modifier, a stabilizer, a preservative, a surfactant, a bulking agent, a flavorant, or any combination thereof.

In certain embodiments, the bulking agent is selected from mannitol, trehalose, chitosan, HP-β-Cyclodextrin, hydroxypropylmethylcellulose (HPMC), dextran, dextrose, starch (such as pea starch), and sucrose.

In certain embodiments, the colloid comprises micelles. In certain embodiments, the colloidal suspension comprises liposomes. In certain embodiments, the colloidal suspension comprises nanoparticles. In certain embodiments, the colloidal suspension comprises microparticles.

In certain embodiments, the spray dried powder is reconstituted with water prior to administration to the patient.

In some embodiments, the MPLA compound is administered in a composition comprising one or more pharmaceutically acceptable excipients. The phrase “pharmaceutically acceptable” is art-recognized. In certain embodiments, the term includes compositions, excipients, adjuvants, polymers, and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

For example, the composition comprising the MPLA compound can be formulated for intranasal delivery as a dry powder, as an aqueous solution, an aqueous suspension, a colloid, a water-in-oil emulsion, a micellar formulation, or as a liposomal formulation.

In some embodiments, the MPLA composition comprises micelles of the MPLA compound. While not being bound by theory, it is believed that micelles enhance the activity of the MPLA. The size of the micelles, in some embodiments, is about 50 nm to about 1000 nm. The size of micelles may be measured by various techniques, including dynamic light scattering (DLS), scanning electron microscopy (SEM), and transmission electron microscopy (TEM). Accordingly, in some embodiments, the size of the micelles is about 50 nm to about 1000 nm as measured by DLS.

In certain preferred embodiments, the composition further comprises an organic solvent, such as an alcohol, glycerin, low molecular weight polyethylene glycol, a poloxamer, or any combination thereof. In some embodiments, the organic solvent is water miscible. In some embodiments, the organic solvent is an alcohol, such as methanol, ethanol, isopropanol, or t-butanol, preferably ethanol.

In some embodiments, the composition further comprises a fatty acid salt, fatty acids, a phospholipid, or any combination thereof.

In some embodiments, the composition comprises a phospholipid or a mixture of phospholipids. Examples of phospholipids include, but are not limited to, phosphatidic acid (PA), phosphatidylcholine (PC), phosphatidylglycerol (PG), phophatidylethanolamine (PE), phophatidylinositol (PI), and phosphatidylserine (PS), sphingomyelin (including brain sphingomyelin), lecithin, lysolecithin, lysophosphatidylethanolamine, cerebrosides, diarachidoylphosphatidylcholine (DAPC), didecanoyl-L-alpha-phosphatidylcholine (DDPC), dielaidoylphosphatidylcholine (DEPC), dilauroylphosphatidylcholine (DLPC), dilinoleoylphosphatidylcholine, dimyristoylphosphatidylcholine (DMPC), dioleoylphosphatidylcholine (DOPC), dipalmitoylphosphatidylcholine (DPPC), distearoylphosphatidylcholine (DSPC), 1-palmitoyl-2-oleoyl-phosphatidylcholine (POPC), diarachidoylphosphatidylglycerol (DAPG), didecanoyl-L-alpha-phosphatidylglycerol (DDPG), dielaidoylphosphatidylglycerol (DEPG), dilauroylphosphatidylglycerol (DLPG), dilinoleoylphosphatidylglycerol, dimyristoylphosphatidylglycerol (DMPG), dioleoylphosphatidylglycerol (DOPG), dipalmitoylphosphatidylglycerol (DPPG), distearoylphosphatidylglycerol (DSPG), 1-palmitoyl-2-oleoyl-phosphatidylglycerol (POPG), diarachidoylphosphatidylethanolamine (DAPE), didecanoyl-L-alpha-phosphatidylethanolamine (DDPE), dielaidoylphosphatidylethanolamine (DEPE), dilauroylphosphatidylethanolamine (DLPE), dilinoleoylphosphatidylethanolamine, dimyristoylphosphatidylethanolamine (DMPE), dioleoylphosphatidylethanolamine (DOPE), dipalmitoylphosphatidylethanolamine (DPPE), distearoylphosphatidylethanolamine (DSPE), 1-palmitoyl-2-oleoyl-phosphatidylethanolamine (POPE), diarachidoylphosphatidylinositol (DAPI), didecanoyl-L-alpha-phosphatidylinositol (DDPI), dielaidoylphosphatidylinositol (DEPI), dilauroylphosphatidylinositol (DLPI), dilinoleoylphosphatidylinositol, dimyristoylphosphatidylinositol (DMPI), dioleoylphosphatidylinositol (DOPI), dipalmitoylphosphatidylinositol (DPPI), distearoylphosphatidylinositol (DSPI), 1-palmitoyl-2-oleoyl-phosphatidylinositol (POPI), diarachidoylphosphatidylserine (DAPS), didecanoyl-L-alpha-phosphatidylserine (DDPS), dielaidoylphosphatidylserine (DEPS), dilauroylphosphatidylserine (DLPS), dilinoleoylphosphatidylserine, dimyristoylphosphatidylserine (DMPS), dioleoylphosphatidylserine (DOPS), dipalmitoylphosphatidylserine (DPPS), distearoylphosphatidylserine (DSPS), 1-palmitoyl-2-oleoyl-phosphatidylserine (POPS), diarachidoyl sphingomyelin, didecanoyl sphingomyelin, dielaidoyl sphingomyelin, dilauroyl sphingomyelin, dilinoleoyl sphingomyelin, dimyristoyl sphingomyelin, sphingomyelin, dioleoyl sphingomyelin, dipalmitoyl sphingomyelin, distearoyl sphingomyelin, 1-palmitoyl-2-oleoyl-sphingomyelin, and any combination thereof.

In certain embodiments, the phospholipid is DPPC, DOPC, cholesterol, or a mixture thereof.

In certain embodiments, compositions comprising MPLA may contain pH modifiers, pH buffers, oils/emulsifiers (e.g., squalene), tonicity modifiers, stabilizers, preservatives, detergents, flavorants, bulking agents, or secondary immunostimulatory agents. In some embodiments, the composition is a dry powder comprising a bulking agent.

Secondary immunostimulatory agents include, e.g., gonadocorticoids, deoxycholic acid, vitamin D, and beta-glucans. Suitable buffers include sodium chloride-based or potassium chloride-based solutions such as phosphate buffered saline, potassium buffered saline, calcium chloride, sodium lactate, sodium bicarbonate, or borate buffered saline. In some embodiments, the buffer may contain salts, detergents, or carbohydrates which preserve the MPLA upon drying and aid in resolubilizing the MPLA upon encounter with a liquid. Suitable carbohydrates include trehalose, sucrose, glucose, and mannose.

In some embodiments, the composition further comprises a mucoadhesive. Suitable mucoadhesives include: glycosylaminoglycans (GAGS) including chondroitin sulfate, chitosan, hyaluronic acid, cellulose derivatives, HP-B-Cyclodextrin, polyacrylates, starch, HPMC and any combination thereof.

In some embodiments, the mucoadhesive is present in the composition in an amount ranging from about 0.1 to about 50% by weight, about 25% to about 50% by weight, or about 49% by weight.

In some embodiments, the composition further comprises a sugar. Examples of sugars that may be used in the methods provided herein include, but are not limited to, dextrose, sucrose, glucose, fructose, lactose, maltose, mannose, galactose, trehalose, and combinations thereof. In certain embodiments, the sugar content is about 49% by weight.

In some embodiments, the composition is an aqueous liquid. In such embodiments, the concentration of the MPLA compound in the composition may be about 1 μg/mL to about 1000 μg/mL, about 20 μg/mL to about 500 μg/mL, about 100 μg/mL to about 300 μg/mL, or about 250 μg/mL. In other such embodiments, the concentration of the MPLA compound in the composition may about 0.01 μg/mL to about 1000 μg/mL, about 0.2 μg/mL to about 20 μg/mL, about 0.5 μg/mL to about 10 μg/mL. In certain embodiments, the concentration is 2.5 μg/mL, and in still other embodiments, the concentration is 1.25 μg/mL.

In certain embodiments, the formulation may contain ionic or nonionic surfactants. Suitable surfactants include poloxamer 407, poloxamer 181, dodecyltrimethylammonium bromide (DTAB), n-dodecyl octaethylene oxide (C12E8), n-dodecyl tetraethylene oxide (C12E4) and dioctanoyl phosphatidylcholine (C8-lecithin), polyoxyl 35 castor oil, cremophor EL (CrEL), octaethylene glycol monododecyl ether (C12E8), hexadecyltrimethylammonium bromide (CTAB), polypropylene oxide (PPO), polyethylene oxide (PEO), PEO-poly(D,L-lactic acid-co-caprolactone) (PEO-PDLLA), and sodium dodecyl sulfate (SDS) and any combination thereof.

In some embodiments, the MPLA formulation has a pH between 4 and 9. In certain preferred embodiments, the pH is between 5 and 8.

In certain embodiments, the formulations may be free of or substantially free of phospholipids, surfactants, salt (e.g., NaCl), and/or buffers. Substantially free means that the substance in question makes up less than 0.5%, less than 0.25%, less than 0.1%, less than 0.05%, less than 0.01%, or less than 0.005% of the composition by weight.

In some embodiments of the invention, the composition comprises an MPLA compound at a concentration between 1 and 8000 μg/mL. In certain preferred embodiments, the MPLA is present at a concentration between 20 and 500 μg/mL. In certain more preferred embodiments, the concentration is between 100 and 300 μg/mL. In certain embodiments, the concentration is 250 μg/mL, and in still other embodiments, the concentration is 125 μg/mL. In some embodiments of the invention, the composition comprises an MPLA compound at a concentration between 0.01 and 1000 μg/mL. In certain preferred embodiments, the MPLA is present at a concentration between 0.2 and 20 μg/mL. In certain more preferred embodiments, the concentration is between 0.5 and 10 μg/mL. In certain embodiments, the concentration is 2.5 μg/mL, and in still other embodiments, the concentration is 1.25 μg/mL.

In some embodiments of the invention, the solution is formulated such that a surfactant is included at a concentration between 1 and 40% w/w, which can enhance the absorption of the drug upon administration by preventing degradation/metabolism, enhancing barrier permeability via transient opening of tight junctions, disruption of lipid bilayer packing/complexation/carrier/ion pairing and enhancing resident time/slowing down mucociliary clearance. In certain preferred embodiments, the surfactant concentration is between 1 and 25% w/w. In certain most preferred embodiments, the surfactant concentration is 15% w/w. Surfactants of interest include, but are not limited to, dipalmitoyl phosphatidyl choline, soybean lecithin, phosphatidylcholine, sodium taurocholate, sodium deoxycholate sodium, glycodeoxycholate, palmitic acid, stearic acid, and oleic acid.

In some embodiments of the invention, the composition comprises a mucoadhesive at a concentration between 0.1 to 50% w/w, which can enhance the absorption of the drug upon administration by enhancing resident time and/or slowing down mucociliary clearance. In certain preferred embodiments, the mucoadhesive is included at a concentration between 40 to 50% w/w. In certain most preferred embodiments, the mucoadhesive is included at a concentration of 49% w/w. Mucoadhesives of interest include, but are not limited to, cellulose derivatives, HP-β-Cyclodextrin polyacrylates, starch, and chitosan.

In accordance with the invention, the MPLA compound can be administered intranasally in low to high doses as an adjuvant to AIT. In some embodiments of the invention, the MPLA is formulated at a concentration between 0.01 and 1000 micrograms/mL, or about 1 and 800 micrograms/mL. In certain preferred embodiments, the MPLA is formulated at a concentration between 1 and 50 micrograms/mL. In more preferred embodiments, the concentration is between 25 and 300 micrograms/mL, and in even more preferred embodiments, between 2.5 and 20 micrograms/mL. In particularly preferred embodiments, the concentration is 2.5 micrograms/mL or 5 micrograms/mL. The total dose given may be divided in equal or unequal parts between both nostrils or a dose may be defined as 1 puff into 1 nostril or 1 puff into both nostrils.

In some embodiments, the AIT treatment is for a food allergy, animal dander, feathers, pollen, or insect venom. Pollen allergies include seasonal allergic rhinitis from grass, tree or weed pollens. Animal dander allergies can be from any animal, preferably dogs, cats and/or horses. Insect venom, in some embodiments, is hymenoptera (e.g., yellow jackets, hornets, wasps, and bees) venom. In preferred embodiments, the allergy is a food allergy. Food allergies can encompass any food a subject is allergic to. In preferred embodiments, the food allergy is peanut, tree nut, sesame seed, dairy, fish, shellfish, wheat, soy, egg, and combinations thereof. In particularly preferred embodiments, the food allergy is peanut.

### Administration

The MPLA compound for the can be administered systemically or directly to the mucosa (topically).

Methods of systemic delivery include those methods known in the art that provide delivery of the active molecule (e.g., the drug) to the circulatory system with distribution throughout the body. Systemic delivery methods include intramuscular, intravenous, intrathecal, subcutaneous, intraperitoneal, sublingual, and oral. As will be understood, any method of systemic delivery is suitable for use with the invention. Particularly suitable methods of systemic delivery include intramuscular and intravenous delivery.

Methods for mucosal delivery include those methods known in the art that provide delivery of the active molecule (e.g., the drug or immunostimulatory agent) to mucous membranes. Mucosal delivery methods include intranasal, intratracheal, intrabuccal, intravaginal, intrarectal, sublingual, and oral. In certain most preferred embodiments, the method of delivery is topical intranasal administration.

In some embodiments, the MPLA compound is administered intranasally. In these embodiments, the MPLA compound may be formulated for delivery to the nasal passages or nasal vestibule of the subject as droplets, an aerosol, micelles, lipid or liquid nanospheres, lipid or liquid microspheres, liposomes, a solution spray, reconstituted powder, or a powder. The composition can be administered by direct application to the nasal passages or may be atomized or nebulized for inhalation through the nose or mouth. In some embodiments, the nasal spray or drops may be accomplished by time of use reconstitution of the product powder with an aqueous vehicle immediately prior to administration. In some embodiments, the nasal spray or drops may be accomplished by reconstitution of the solid drug product powder in a suitable delivery device using an aqueous vehicle some period in which the drug product is deemed stable in solution format prior to administration.

In some embodiments, administering comprises administration of a nasal spray, medicated nasal swab, medicated wipe, liquid drops, or aerosol to the subject's nasal passages or nasal vestibule.

In some embodiments, administering comprises of a combination of the aforementioned administration methods.

### EXAMPLES

In order that the invention described herein may be more fully understood, the following examples are set forth. The examples described in this application are offered to illustrate the compounds, compositions, materials, device, and methods provided herein and are not to be construed in any way as limiting their scope.

### Example 1

### Preparation of PHAD Micelles in 5% Ethanol.

1 mg of PHAD was wetted for 1 minute in 0.4 mL of 95% ethanol then sonicated for 15 minutes at 40° C. until a clear solution is formed. The solution was removed from the sonication bath and QS to 8 mL with water, resulting in a homogeneous formulation of PHAD micelles 150 nm or smaller in size. The formulation was either used as liquid or lyophilized.

### Example 2

### Preparation of PHAD Powder.

6 mg of PHAD was wetted and dissolved in 3 mL of 95% ethanol at 40° C. and sonicated for 20 minutes at 40° C. until a clear solution of 2 mg/mL PHAD was obtained. 17 mL of water at 40° C. was then added and sonicated to fully mix the bulk solution, resulting in a homogeneous formulation of PHAD micelles around 150 nm or smaller in size. 147 mg of HP-β-Cyclodextrin was added to the solution under mixing. This solution was then spray dried to obtain a final powder at 10% w/w/w PHAD:HP-β-Cyclodextrin. This particular composition of PHAD was readily soluble in water at concentrations up to 8 mg/mL. This solution was then diluted into 5% dextrose, and sterile filtered for intravenous administration.

### Example 3

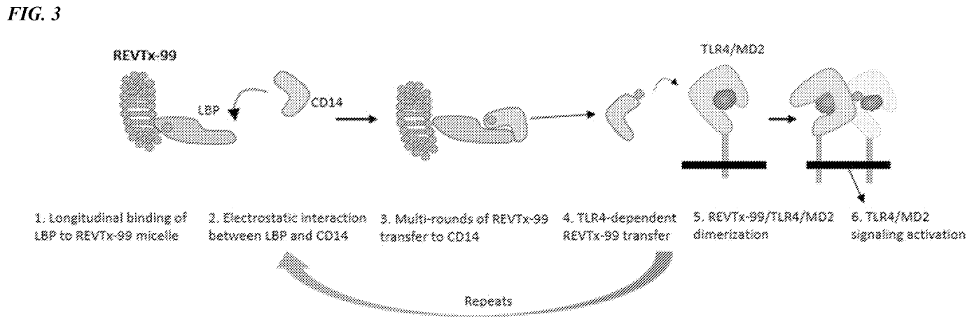

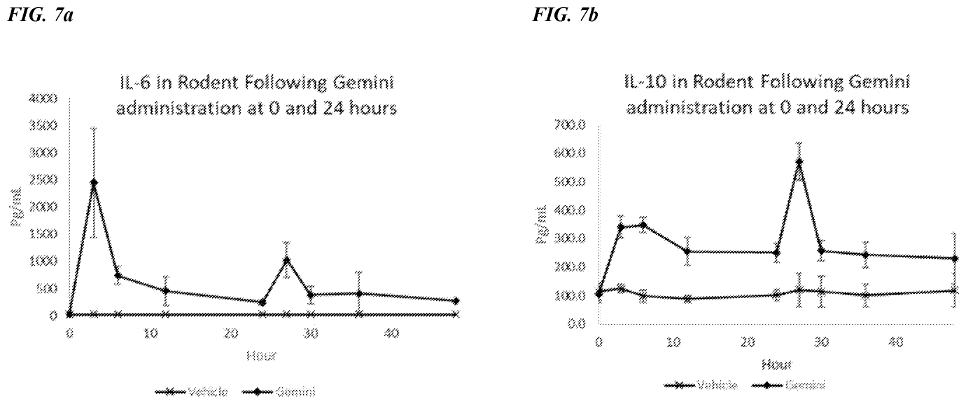

Formulations as prepared in Examples 1 and 2 show robust upregulation of IL-10 and IL-6 as a result of IV administration in rats (FIGS. 7a and 7b). IL-6 and IL-10 are important cytokines that demonstrate TLR4 stimulation. Formulations as prepared in Examples 1 and 2 show robust upregulation of IP-10, as a result of TLR4 stimulation in vitro in mouse macrophages (FIG. 3). IP-10 is an important cytokine associated with stimulation of the TRIF pathway. Evidence of IL-6 and IL-10 and IP-10 upregulation is confirmation that PHAD preparations as noted in this application are capable of TLR4 stimulation, with a bias toward stimulating the TRIF pathway, which is further confirmation of the amelioration or reduction of activity mediated through the MyD88 signaling pathway, which supports the amelioration of pro-inflammatory cytokine production.

### Example 4

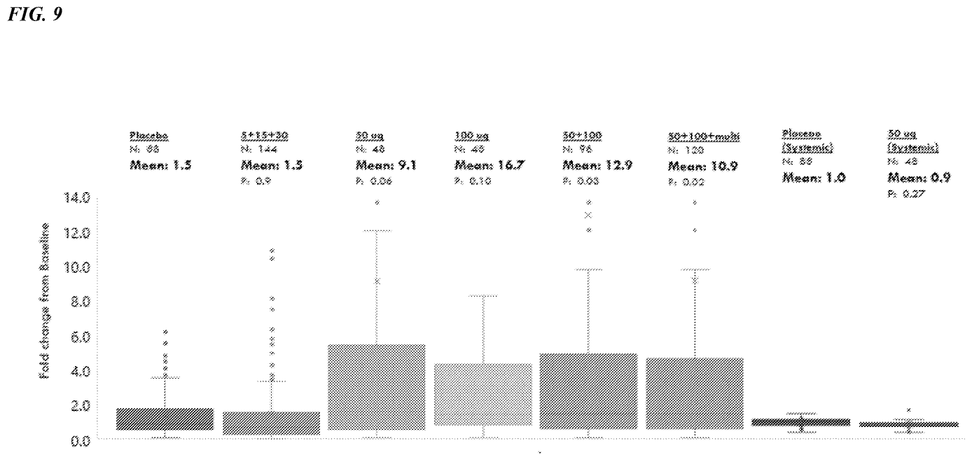

Formulations as prepared in Example 1 show a dose dependent upregulation of IP-10 when administered intranasally to healthy human volunteers (FIG. 9). The Formulation of Example 1 did not stimulate a systemic IP-10, and PK data shows no systemic exposure above 5 pg/mL limit of quantitation, supporting the premise that the exemplary formulation of PHAD acts locally. Presented is a box and whisker plot of the fold-change values for all data and time-points including the mean fold-change and p-value calculated using a t-test comparing placebo to each data set presented.

### Example 5

### Formulation of PHAD for Intranasal Administration at 1/mL

| Material | Amount | |
|---|---|---|
| PHAD | 1 | μg |
| Trehalasoe | 199.5 | mg |
| Water for irrigation | 1 | mL |

### Example 6

Formulation of PHAD for Intravenous Administration at 1 μg/mL

| Material | Amount | |
|---|---|---|
| PHAD | 1 | μg |
| HP-β-cyclodextrin | 9.0 | ug |
| Water for irrigation | 0.1 | mL |
| 5% Dextrose | 0.9 | mL |

### Example 7

Proof-of-concept clinical study to evaluate safety and tolerability of PHAD as an adjunct to oral peanut AIT. The study will employ an open-label crossover design in which subjects will be randomized into either the pre-treatment cohort or the posttreatment cohort. Adult (18 to 60 years of age) subjects with a history of hypersensitivity allergy to peanuts will be screened for adequate peanut allergy by serum IgE to peanuts (≥10 kUA/L) and a skin prick to peanuts (≥8 mm compared to a negative control). Adults with history of disease(s) (heart disease, diabetes, hypertension) that would represent a risk to the subject's health or safety or ability to comply with the protocol will not be eligible for the study. In addition, other key exclusionary may include the following: systemic corticosteroid use within 6 months prior to screening, topical corticosteroid use with 28 days prior to screening, and/or use of drugs that are known or likely to interact with epinephrine (e.g., beta-blockers, angiotensin converting enzyme-inhibitors, tri-cyclic antidepressants, or other drugs), within 3 weeks prior to screening. Eligible subjects will undergo a screening/baseline oral peanut challenge test to assess baseline signs, symptoms, serum IgE, and intranasal IgA. After an adequate washout period, subjects will participate in a 1-day treatment period that will include a repeat oral peanut allergy challenge test. Subjects randomized to the pre-treatment cohort will receive a single dose intranasally (1 puff each nostril) of a composition containing 50-150 micrograms of PHAD prior to the oral peanut challenge, and subjects randomized to the post-treatment cohort will receive a single dose of 50-150 micrograms after the oral peanut challenge. The primary endpoint will be change in allergic symptoms (scale of 1 [not bad] to 10 [terribly bad]) as measured by mean intrasubject change by cohort in allergy severity symptom score between baseline (screening challenge) and Day 1. Other key endpoints will include incidence in use of rescue therapy compared to baseline, percent change in serum IgE and nasal IgA compared to baseline, and, in the pre-treatment cohort only, change in cumulative tolerated dose of peanut protein compared to baseline. After the 1 day treatment period, subjects will complete a safety follow-up televisit on Day 30.

### Example 8

Proof-of-concept clinical study to evaluate safety and tolerability of PHAD as an adjunct to oral peanut AIT. The study will employ an open-label crossover design in which subjects will be randomized into either the pre-treatment cohort or the posttreatment cohort. Adult (18 to 60 years of age) subjects with a history of hypersensitivity allergy to peanuts will be screened for adequate peanut allergy by serum IgE to peanuts (≥10 kUA/L) and a skin prick to peanuts (≥8 mm compared to a negative control). Adults with history of disease(s) (heart disease, diabetes, hypertension) that would represent a risk to the subject's health or safety or ability to comply with the protocol will not be eligible for the study. In addition, other key exclusionary may include the following: systemic corticosteroid use within 6 months prior to screening, topical corticosteroid use with 28 days prior to screening, and/or use of drugs that are known or likely to interact with epinephrine (e.g., beta-blockers, angiotensin converting enzyme-inhibitors, tri-cyclic antidepressants, or other drugs), within 3 weeks prior to screening. Eligible subjects will undergo a screening/baseline oral peanut challenge test to assess baseline signs, symptoms, serum IgE, and intranasal IgA. After an adequate washout period, subjects will participate in a 1-day treatment period that will include a repeat oral peanut allergy challenge test. Subjects randomized to the pre-treatment cohort will receive a single dose intravenously or subcutaneous of a composition containing 50-150 micrograms of PHAD prior to the oral peanut challenge, and subjects randomized to the post-treatment cohort will receive a single dose of 50-150 micrograms after the oral peanut challenge. The primary endpoint will be change in allergic symptoms (scale of 1 [not bad] to 10 [terribly bad]) as measured by mean intrasubject change by cohort in allergy severity symptom score between baseline (screening challenge) and Day 1. Other key endpoints will include incidence in use of rescue therapy compared to baseline, percent change in serum IgE and nasal IgA compared to baseline, and, in the pre-treatment cohort only, change in cumulative tolerated dose of peanut protein compared to baseline. After the 1 day treatment period, subjects will complete a safety follow-up televisit on Day 30.

### Example 9

Other studies conducted in parallel or later phase may employ a variety of design modalities such as randomized, double-blind, placebo-controlled trials in adults and/or children.

Dosing: Examples include peri-AIT (before and/or after AIT treatment); administered as a single intranasal dose within 3 days prior to AIT therapy, immediately before or after AIT treatment course, or up to 24 hours after AIT treatment course.

Results: PHAD compositions will be considered efficacious if one or more of the following are met:
Superiority or non-inferiority of PHAD compositions to control in the proportion of subjects who “pass” a post-baseline allergen challenge test;
Superiority or non-inferiority of PHAD compositions to control in the proportion of subjects who pass a maintenance phase allergen challenge test;
Superiority or non-inferiority of PHAD compositions to control in change (percent, mean, median, or absolute value) in cumulative tolerated dose of allergen during a post-baseline allergen challenge test;
Superiority or non-inferiority of PHAD compositions to control in proportion of subjects who reach the protocol-defined maximum dose of allergen
Superiority or non-inferiority of PHAD compositions to control in time to reach the protocol defined maximum dose of allergen; Superiority or non-inferiority of PHAD compositions to control in change (percent, mean, median, or absolute value) in cumulative tolerated maintenance dose of allergen;
Superiority or non-inferiority of PHAD compositions to control in time to maintenance phase of AIT; Superiority or non-inferiority of PHAD compositions to control in change (percent, mean, median, or absolute value) in allergen-specific and/or total serum IgE; Superiority or non-inferiority of PHAD compositions to control in change (percent, mean, median, or absolute value) in IgG (and all subclasses), IgA (and both subclasses), IgM, and/or IgD.
Superiority or non-inferiority of PHAD compositions to control in overall, weekly, or daily mean, median, maximum, or absolute severity of allergen symptoms (e.g., on a scale of 0 [absent] to 12 [maximum severity], 1 [not bad] to 10 [terribly bad]);
Superiority or non-inferiority of PHAD compositions to control in change (percent, mean, median, or absolute value) in blood basophil histamine release test, serum mass cell tryptase, objective airway tests (e.g., peak inhalation test, forced 1 second breath (FEV1) test), or skin prick tests;
Superiority or non-inferiority of PHAD compositions to control in change (percent, mean, median, or absolute value) in relevant cytokines and chemokines (e.g., IL-4, IL-5, IL-6, IL-8, IL-9, IL-10, IL-12, IL 13, IL-22, IL-25, IL-33, IFN-α, IFN-β, IFN-γ, interferon gamma-induced protein (IP-10), ECP, TGF β, or VEGF), or other mediators and constituents which may be assessed such as eosinophils, histamine, eotaxins, or leukotriene.

Endpoints may be measured from baseline up to the point of an immediate change in symptoms (e.g., within 5 minutes) or up to the time it takes for the subject to meet the protocol-specified maintenance phase (e.g., up to 40 weeks, up to 76 weeks). Where applicable, laboratory endpoints may be analyzed by blood-, saliva-, urine-, or nasal-derived specimens where appropriate. Control may include placebo study drug, no study drug, or active comparator study drug.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A method of adjuvant treatment in a subject undergoing allergy immunotherapy (AIT) comprising administering to a subject in need thereof a monophosphoryl lipid A (MPLA) compound.

2. The method of claim 1, wherein the MPLA compound is administered to the mucosa of the subject.

3. The method of claim 1 or 2, wherein the MPLA compound is administered intranasally.

4. The method of any one of claim 1, wherein the MPLA compound is administered systemically to the subject.

5. The method of claim 1, wherein the MPLA compound is administered intravenously, subcutaneously, intrathecally, orally, sublingually, or intramuscularly.

6. The method claim 5, wherein the MPLA compound is administered intravenously.

7. The method of any one of claims 1 to 6, wherein the MPLA is administered in a dose from about 1.0 mcg to about 1000 mcg.

8. The method of claim 7, wherein the MPLA is administered in a dose from about 1 mcg to about 1000 mcg.

9. The method of claim 8, wherein the MPLA is administered in a dose from about 10 mcg to about 500 mcg.

10. The method of claim 9, wherein the MPLA is administered in a dose of about 200 mcg.

11. The method of any one of claims 1 to 10, wherein half of the dose of MPLA is administered to each nasal passage of the subject.

12. The method of any one of claims 1 to 10, wherein the entire dose of MPLA is administered to a single nasal passage of the subject.

13. The method any one of claims 1 to 12, wherein the MPLA compound is administered within 3 days prior to AIT treatment or within 24 hours after AIT treatment course.

14. The method of any one of claims 1 to 13, wherein the MPLA compound wherein the MPLA compound is selected from phosphorylated hexaacyl disaccharide (PHAD), PHAD-504, 3D (6-acyl)-PHAD, 3D-PHAD and any combination thereof.

15. The method of claim 14, wherein the MPLA compound is PHAD.

16. The method of any one of claims 1 to 12, wherein the composition further comprises a sugar.

17. The method of claim 16, wherein the sugar is chosen from a monosaccharide, a disaccharide, a trisaccharide, a linear oligosaccharide, a branched oligosaccharide, a cyclic oligosaccharide, a linear polysaccharide, a branched polysaccharide, and any combination thereof.

18. The method of claim 17, wherein the monosaccharide is selected from glucose, dextrose, fructose, galactose, xylose, ribose, and any combination thereof.

19. The method of claim 17 or 18, wherein the disaccharide is selected from trehalose, sucrose, maltose, lactose, and any combination thereof.

20. The method of any one of claims 17 to 19, wherein the trisaccharide is selected from nigerotriose, maltotriose, melezitose, maltotriulose, raffinose, kestose, and any combination thereof.

21. The method of any one of claims 17 to 20, wherein the linear or branched oligosaccharide is selected from nigerotetraose, maltotetraose, lychnose, nystose, sesamose, stachyose.

22. The method of any one of claims 17 to 21, wherein the cyclic oligosaccharide is selected from alpha-cyclodextrin, beta-cyclodextrin, or gamma-cyclodextrin.

23. The method of any one of claims 17 to 22, wherein the linear or branched polysaccharide is selected from starch, glucan, chitosan, pectin, carboxymethyl cellulose, glycosylaminoglycans, hyaluronic acid, cellulose derivatives, hydroxypropylmethylcellulose (IPMC), dextran, and any combination thereof.

24. The method of any one of claims 17 to 23, wherein the disaccharide is trehalose.

25. The method of any one of claims 17 to 24, wherein the cyclic oligosaccharide is beta-cyclodextrin.

26. The method of any one of claims 1 to 25, wherein the composition further comprises a surfactant selected from poloxamer 407, poloxamer 181, dodecyltrimethylammonium bromide (DTAB), n-dodecyl octa (ethylene oxide) (C12E8), n-dodecyl tetra (ethylene oxide) (C12E4), dioctanoyl phosphatidylcholine (C8-lecithin), Polyoxyl 35 castor oil, Cremophor EL (CrEL), Octaethylene glycol monododecyl ether (C12E8), hexadecyltrimethylammonium bromide (CTAB), polypropylene oxide (PPO), polyethylene oxide (PEO), PEO-poly(D,L-lactic acid-co-caprolactone) (PEO-PDLLA), sodium dodecyl sulfate (SDS), triethylamine, trimethylamine, and any combination thereof.

27. The pharmaceutical composition of any one of claims 1 to 26, wherein the composition further comprises a phospholipid selected from phosphatidic acid (“PA”), phosphatidylcholine (“PC”), phosphatidylglycerol (“PG”), phophatidylethanolamine (“PE”), phophatidylinositol (“PI”), phosphatidylserine (“PS”), sphingomyelin (including brain sphingomyelin), lecithin, lysolecithin, lysophosphatidylethanolamine, cerebrosides, diarachidoylphosphatidylcholine (“DAPC”), didecanoyl-L-alpha-phosphatidylcholine (“DDPC”), dielaidoylphosphatidylcholine (“DEPC”), dilauroylphosphatidylcholine (“DLPC”), dilinoleoylphosphatidylcholine, dimyristoylphosphatidylcholine (“DMPC”), dioleoylphosphatidylcholine (“DOPC”), dipalmitoylphosphatidylcholine (“DPPC”), distearoylphosphatidylcholine (“DSPC”), 1-palmitoyl-2-oleoyl-phosphatidylcholine (“POPC”), diarachidoylphosphatidylglycerol (“DAPG”), didecanoyl-L-alpha-phosphatidylglycerol (“DDPG”), dielaidoylphosphatidylglycerol (“DEPG”), dilauroylphosphatidylglycerol (“DLPG”), dilinoleoylphosphatidylglycerol, dimyristoylphosphatidylglycerol (“DMPG”), dioleoylphosphatidylglycerol (“DOPG”), dipalmitoylphosphatidylglycerol (“DPPG”), distearoylphosphatidylglycerol (“DSPG”), 1-palmitoyl-2-oleoyl-phosphatidylglycerol (“POPG”), diarachidoylphosphatidylethanolamine (“DAPE”), didecanoyl-L-alpha-phosphatidylethanolamine (“DDPE”), dielaidoylphosphatidylethanolamine (“DEPE”), dilauroylphosphatidylethanolamine (“DLPE”), dilinoleoylphosphatidylethanolamine, dimyristoylphosphatidylethanolamine (“DMPE”), dioleoylphosphatidylethanolamine (“DOPE”), dipalmitoylphosphatidylethanolamine (“DPPE”), distearoylphosphatidylethanolamine (“DSPE”), 1-palmitoyl-2-oleoyl-phosphatidylethanolamine (“POPE”), diarachidoylphosphatidylinositol (“DAPI”), didecanoyl-L-alpha-phosphatidylinositol (“DDPI”), dielaidoylphosphatidylinositol (“DEPI”), dilauroylphosphatidylinositol (“DLPI”), dilinoleoylphosphatidylinositol, dimyristoylphosphatidylinositol (“DMPI”), dioleoylphosphatidylinositol (“DOPI”), dipalmitoylphosphatidylinositol (“DPPI”), distearoylphosphatidylinositol (“DSPI”), 1-palmitoyl-2-oleoyl-phosphatidylinositol (“POPI”), diarachidoylphosphatidylserine (“DAPS”), di decanoyl-L-alpha-phosphatidylserine (“DDPS”), dielaidoylphosphatidylserine (“DEPS”), dilauroylphosphatidylserine (“DLPS”), dilinoleoylphosphatidylserine, dimyristoylphosphatidylserine (“DMPS”), dioleoylphosphatidylserine (“DOPS”), dipalmitoylphosphatidylserine (“DPPS”), distearoylphosphatidylserine (“DSPS”), 1-palmitoyl-2-oleoyl-phosphatidylserine (“POPS”), diarachidoyl sphingomyelin, didecanoyl sphingomyelin, dielaidoyl sphingomyelin, dilauroyl sphingomyelin, dilinolcoyl sphingomyelin, dimyristoyl sphingomyelin, sphingomyelin, dioleoyl sphingomyelin, dipalmitoyl sphingomyelin, distearoyl sphingomyelin, 1-palmitoyl-2-oleoyl-sphingomyelin, and any combination thereof.

28. The method of any one of claims 1 to 27, wherein the MPLA compound is between 0.5% and 10% by weight of the composition.

29. The method of any one of claims 1 to 28, wherein the sugar is between 80% and 99.5% by weight of the composition.

30. The method of claims 26 to 29, wherein the surfactant is between 0.5% and 10% by weight of the composition.

31. The method of any one of claims 27 to 30, wherein the phospholipid is between 0.5% and 10% by weight of the composition.

32. The method of any one of claims 1 to 31, wherein the composition is a dry powder.

33. The method of any one of claims 1 to 31, wherein the composition is an aqueous composition.

34. The method of claim 33, further comprising an organic solvent.

35. The method of claim 34, wherein the organic solvent and water are present in a volume to volume ratio of about 1:1500 to about 1:50.

36. The method of claim 35, wherein the organic solvent and water are present in a volume to volume ratio of about 1:1000 to about 1:100.

37. The method of claim 36, wherein the organic solvent and water are present in a volume to volume ratio of about 1:800.

38. The method of any one of claims 34 to 37, wherein the organic solvent is miscible with water.

39. The method of any one of claims 34 to 38, wherein the organic solvent is selected from an alcohol, glycerin, low molecular weight polyethylene glycol, and low molecular weight poloxamers.

40. The method of claim 39, wherein the organic solvent is an alcohol.

41. The method of claim 40, wherein the alcohol is selected from methanol, ethanol, isopropanol, or t-butanol.

42. The method of claim 38, wherein the alcohol is ethanol.

43. The method of any one of claims 33 to 42, wherein the composition has an MPLA compound concentration of about 1 μg/mL to about 1000 μg/mL.

44. The method of claim 43, wherein the composition has a MPLA compound concentration of about 0.2 μg/mL to about 20 μg/mL.

45. The method of claim 44, wherein the composition has a MPLA compound concentration of about 1.25 μg/mL.

46. The method of claim 45, wherein the composition has a MPLA concentration of about 2.5 μg/mL.

47. The method of any one of claims 1 to 46, wherein the composition further comprises a stabilizer.

48. The method of any one of claims 1 to 47, wherein the composition comprises micelles having an average diameter or length of about 1 nm to about 1000 nm.

49. The method of claim 48, wherein the composition comprises micelles having an average diameter or length of about 50 nm to about 500 nm.

50. The method of claim 49, wherein the composition comprises micelles having an average diameter or length of about 100 nm to about 500 nm.

51. The method of any one of claims 1 to 50, further comprising a mucoadhesive agent selected from cellulose derivatives, polyacrylates, a starch, chitosan, glycosylaminoglycans, hyaluronic acid, cellulose derivatives, and any combination thereof.

52. The method of any one of claims 1 to 51, further comprising a pH modifier, a pH buffer, an emulsifier, a tonicity modifier, a stabilizer, a preservative, a surfactant, a bulking agent, a flavorant, or any combination thereof.

53. The method of claim 52, wherein the bulking agent is selected from mannitol, trehalose, chitosan, hydroxypropylmethylcellulose (HPMC), dextran, dextrose, pea starch, and sucrose.

54. The method of any one of claims 1 to 53, wherein the composition comprises micelles.

55. The method of any one of claims 1 to 54, wherein the composition comprises liposomes.

56. The method of any one of claims 1 to 55, wherein the composition comprises nanoparticles.

57. The method of any one of claims 1 to 56, wherein the composition comprises microparticles.

58. The method of any one of claims 1 to 54, wherein the composition is administered to the subject intranasally.

59. The method of any one of claims 1 to 58, wherein the composition delivers a total dose of from 1 to 1000 micrograms of MPLA compound to the subject.

60. The method of any one of claims 1 to 59, wherein the MPLA compound is administered within 3 days prior to an AIT treatment.

61. The method of any one of claims 1 to 60, wherein the MPLA compound is administered within about 24 hours after an AIT treatment course.

62. The method of any one of claims 1 to 61, wherein the AIT treatment is for a food allergy, animal dander, feathers, pollen, or insect venom.

63. The method of claim 62, wherein the dander is selected from horse dander, dog dander, cat dander, and any combination thereof.

64. The method of claim 62, wherein the insect venom is hymenoptera venom.

65. The method of claim 62, wherein the food allergy is selected from peanut, tree nut, sesame seed, dairy, fish, shellfish, wheat, soy, egg, and any combination thereof.

66. The method of claim 65, wherein the food allergy is peanut.

67. A method of augmenting the therapeutic action of AIT in a subject in need thereof comprising:
a) determining the subject's response via clinical symptomology using a validated or widely accepted subjective symptom scale;
b) determining the subject's immunoglobulin profile in blood, saliva, or nasal specimens;
c) determining the subject's immunogenic mediator constituents profile in blood, saliva, nasal, or urine specimens;
d) determining the subject's immunogenic effector cell profile in blood, saliva, nasal, or urine specimens;
e) determining the subject's cytokine and/or chemokine profiles in blood or nasal specimens; and
f) determining the subject's objective clinical measures, where the clinical measures comprise peak nasal inspiratory flow, FEV1, hemoglobin A1c, or any combination thereof.

68. The method of claim 67, wherein determining the subject's cytokine, chemokine, effector cells or immunogenic mediator profiles comprises collecting a blood sample via venipuncture, collecting a nasal sample via the subject's nasal passages, collecting a saliva sample via the subject's mouth, or collecting the subject's urinary sample and assaying the sample using a rapid lateral flow assay designed to detect IL-6, IL-4, IL-5, IL-10, IL-13, IFN-α, IFN-β, IFN-γ, TRAIL, or IP-10 or combinations thereof.

69. The method of claim 67 or 68, wherein determining the subject's immunoglobin profile comprises collecting a blood, saliva, or nasal sample to detect IgE, IgG, IgG subclasses, IgA, IgA subclasses, IgM, IgD, or any combination thereof.
